(19) United States
(12) Patent Application Publication
(10) Pub. No.: US 2026/0034203 A1
(43) Pub. Date: Feb. 5, 2026

(54) COMPOSITIONS, KITS, AND METHODS OF IMMUNIZING AGAINST VIRAL INFECTIONS

(71) Applicant: Yale University, New Haven, CT (US)

(72) Inventors: Akiko Iwasaki, New Haven, CT (US); Miyu Moriyama, New Haven, CT (US); Tianyang Mao, New Haven, CT (US); Benjamin Goldman-Israelow, New Haven, CT (US)

(21) Appl. No.: 19/099,104

(22) PCT Filed: Aug. 2, 2023
(86) PCT No.: PCT/US2023/071482

§ 371 Date: Jan. 28, 2025

Related U.S. Application Data
(60) Provisional application No. 63/394,367, filed on Aug. 2, 2022.

(51) Int. Cl. A61K 39/145; A61K 39/00; A61P 31/16; C12N 7/00

(52) U.S. Cl. CPC A61K 39/145; A61P 31/16; C12N 7/00; A61K 2039/543; A61K 2039/544; A61K 2039/545; A61K 2039/572; A61K 2039/575; C12N 2760/16134

(57) ABSTRACT

Described herein is a method of treating, ameliorating, and/or preventing an influenza viral infection in a subject, and/or immunizing a subject against an influenza viral infection, which include parenterally administering to the subject a first composition including a first compound in an amount sufficient to elicit systemic T and/or B cell response(s) against a first influenza virus in the subject; and administering to the subject a second composition including a viral protein of a second influenza virus through an administration route comprising intranasal, inhalational, intratracheal, intrapulmonary, and intrabronchial, in an amount sufficient to result in establishment of tissue-resident T cell(s), tissue-resident B cell(s), mucosal IgA, and/or systemic IgG specific against the influenza viral infection in the subject. Also described are a kit for performing the method, as well as methods and kits for boosting existing immunity against influenza viral infections.

### CROSS-REFERENCE TO RELATED APPLICATIONS

The present application claims priority under 35 U.S.C. § 119 (e) to U.S. Provisional Patent Application No. 63/394,367, filed Aug. 2, 2022, which is incorporated herein by reference in its entirety.

### STATEMENT REGARDING FEDERALLY SPONSORED RESEARCH

This invention was made with government support under 75N93019C00051 awarded by the National Institutes of Health. The government has certain rights in the invention.

### BACKGROUND

It is estimated that in a typical year 5-15% of the world population is infected by the influenza virus. Among those, 3-5 million people experience severe symptoms and up to 650,000 people die from the infection. Further, influenza virus has caused five pandemics in the last one hundred years, as characterized by higher-than-average infection numbers, severe cases and/or deaths: the 1918 influenza pandemic (the “Spanish flu”) in 1918-1920, the 1957-1958 influenza pandemic (the “Asian flu”), the 1968 influenza pandemic (the “Hong Kong flu”), the 1977 influenza pandemic (the “Russian flu”), and the 2009 swine flu pandemic.

Vaccination is considered the most effective way to prevent flu infections, and/or minimize or prevent severe symptoms and/or deaths. Currently, the vast majority of flu vaccines are inactivated virus-based vaccines for parental administration, although a live-attenuated intranasal influenza vaccine (FluMist) is also available.

The effectiveness of the available flu vaccines depends critically on the accurate predictions of the season's influenza variants. The current influenza vaccines are specific to only a few pre-selected viral strains and are sensitive to mismatches. If a subject is administered with flu vaccine against one strain and the season's influenza is caused by a different viral strain, the effectiveness of the vaccine can be greatly reduced. This mismatch sensitivity is highly undesirable as influenza virus has high degree of diversity. The genome of influenza virus consists of eight negative-sense, single-stranded RNA molecules (“segments”). Variants of virus can be generated by reassortment of RNA segments, by recombination of each RNA molecule which is universal to RNA viruses, and by point mutations due to the low fidelity of the reverse-transcription of the RNA molecules. For these reasons, numerous influenza strains are known to exist, and new strains are emerging constantly, which makes accurate prediction of season's influenza difficult.

Furthermore, most of the available flu vaccines can be effective at preventing severe symptom and death when correctly matched with the season's variants, but are much less effective at preventing the transmission of the virus from one subject to another.

Therefore, there is a need to develop novel influenza vaccines and/or immunization strategies that are less sensitive to strain mismatches and/or more effective at preventing viral transmission. The present invention addresses this need.

### SUMMARY

In some aspects, the present invention is directed to the following non-limiting embodiments:

In some aspects, the present invention is directed to a method of immunizing a subject against an influenza viral infection. In some aspects, the present invention is directed to a method of treating, ameliorating, and/or preventing an influenza viral infection in a subject.

In some embodiments, the method comprises parenterally administering to the subject a first composition comprising a first compound in an amount sufficient to elicit systemic T and/or B cell response(s) against a first influenza virus in the subject.

In some embodiments, the method further comprises administering to the subject a second composition comprising a viral protein of a second influenza virus through an administration route comprising intranasal, inhalational, intratracheal, intrapulmonary, and intrabronchial, in an amount sufficient to result in establishment of tissue-resident T and/or B cell(s), mucosal IgA, and/or systemic IgG specific against the influenza viral infection in the subject.

In some embodiments, the method further comprises parentally administering to the subject a third composition for boosting the immune response elicited by the first composition or the immune response elicited by both the first composition and the second composition.

In some embodiments, the third composition is administered between the administrations of the first composition and the second composition, or is administered after the second composition in the subject.

In some embodiments, the third composition is the same as the first composition.

In some embodiments, the first influenza virus or the second influenza virus is independently an influenza A virus, an influenza B virus, an influenza C virus, or combinations thereof.

In some embodiments, a hemagglutinin protein of the first influenza virus or the second influenza virus is independently an H1 subtype, an H2 subtype, an H3 subtype, an H4 subtype, an H5 subtype, an H6 subtype, an H7 subtype, an H8 subtype, an H9 subtype, an H10 subtype, an H11 subtype, an H12 subtype, an H13 subtype, an H14 subtype, an H15 subtype, an H16 subtype, an H17 subtype, an H18 subtype, or combinations thereof.

In some embodiments, a neuraminidase protein of the first influenza virus or the second influenza virus is independently an N1 subtype, an N2 subtype, an N3 subtype, an N4 subtype, an N5 subtype, an N6 subtype, an N7 subtype, an N8 subtype, an N8 subtype, an N9 subtype, an N10 subtype, an N11 subtype, or combinations thereof.

In some embodiments, the first composition and/or the third composition comprises a vaccine against the first influenza virus.

In some embodiments, the vaccine against the first influenza virus comprises at least one selected from the group consisting of a mRNA-lipid nanoparticle (LNP)-based vaccine against the influenza viral infection, a viral vector vaccine against the influenza viral infection, an inactivated virus vaccine against the influenza viral infection, a viral subunit/peptide vaccine against the influenza viral infection, a virus like particle (VLP)-based vaccine against the influenza viral infection, and a DNA vaccine against the influenza viral infection.

In some embodiments, the second composition comprises a surface protein of the second influenza virus.

In some embodiments, the second composition comprises a hemagglutinin protein of the second influenza virus, a neuraminidase protein of the second influenza virus, or combinations thereof.

In some embodiments, the first composition elicits systemic T and/or B cell response(s) against a protein in the first influenza virus the same as or orthologous to the viral protein comprised in the second composition in the subject.

In some embodiments, the viral protein of the second influenza virus in the second composition is a recombinant protein, a purified protein, or both.

In some embodiments, the first influenza virus and the second influenza virus are the same.

In some embodiments, the first influenza virus and the second influenza virus are different.

In some embodiments, the first influenza virus and the second influenza viruses are of the same subtype but different strains.

In some embodiments, the first influenza virus and the second influenza viruses are of different subtypes.

In some embodiments, the second composition does not comprise an adjuvant.

In some embodiments, the second composition comprises a pharmaceutically acceptable carrier suitable for intranasal, inhalational, intratracheal, intrapulmonary, and/or intrabronchial administration.

In some embodiments, the administration of the first composition, the administration of the second composition, and/or the administration of the third composition are separated by about 3 days to about 60 days from each other.

In some embodiments, the method results in expansion of antigen-specific, tissue-resident memory T and/or B cell response(s) against the virus in the lung, the airway or the mediastinal lymph nodes (mLNs) of the subject.

In some embodiments, the subject is a mammal. In some embodiments, the subject is a human.

In some aspects, the present invention is directed to a kit for immunizing a subject against an influenza viral infection.

In some aspects, the present invention is directed to a kit for treating, ameliorating, and/or preventing infection by an influenza virus in a subject.

In some embodiments, the kit comprises a first composition for administering to the subject parenterally, the first composition comprising: a first compound for eliciting systemic T and/or B cell response(s) against a first influenza virus in the subject.

In some embodiments, the kit further comprises a second composition for administering to the subject through an administration route comprising intranasal, inhalational, intratracheal, intrapulmonary, and intrabronchial, the second composition comprising: a viral protein of a second influenza virus for establishment of tissue-resident T and/or B cell(s), mucosal IgA, and/or systemic IgG specific against the influenza viral infection in the subject.

In some embodiments, the kit further comprises a third composition for administering to the subject parenterally, the third composition comprising a compound for boosting the immune response elicited by the first composition or the immune response elicited by both the first composition and the second composition in the subject.

In some embodiments, the third composition is for being administered between the administrations of the first composition and the second composition, or is for being administered after the second composition.

In some embodiments, the third composition is the same as the first composition.

In some embodiments, the first influenza virus or the second influenza virus is independently an influenza A virus, an influenza B virus, an influenza C virus, or combinations thereof.

In some embodiments, a hemagglutinin protein of the first influenza virus or the second influenza virus is independently an H1 subtype, an H2 subtype, an H3 subtype, an H4 subtype, an H5 subtype, an H6 subtype, an H7 subtype, an H8 subtype, an H9 subtype, an H10 subtype, an H11 subtype, an H12 subtype, an H13 subtype, an H14 subtype, an H15 subtype, an H16 subtype, an H17 subtype, an H18 subtype, or combinations thereof.

In some embodiments, a neuraminidase protein of the first influenza virus or the second influenza virus is independently an N1 subtype, an N2 subtype, an N3 subtype, an N4 subtype, an N5 subtype, an N6 subtype, an N7 subtype, an N8 subtype, an N8 subtype, an N9 subtype, an N10 subtype, an N11 subtype, or combinations thereof.

In some embodiments, the first composition and/or the third composition comprises a vaccine against the first influenza virus.

In some embodiments, the vaccine against the first influenza virus comprises at least one selected from the group consisting of a mRNA-lipid nanoparticle (LNP)-based vaccine against the influenza viral infection, a viral vector vaccine against the influenza viral infection, an inactivated virus vaccine against the influenza viral infection, a viral subunit/peptide vaccine against the influenza viral infection, a virus like particle (VLP)-based vaccine against the influenza viral infection, and a DNA vaccine against the influenza viral infection.

In some embodiments, the second composition comprises a surface protein of the second influenza virus.

In some embodiments, the second composition comprises a hemagglutinin protein of the second influenza virus, a neuraminidase protein of the second influenza virus, or combinations thereof.

In some embodiments, the first composition elicits systemic T and/or B cell response(s) against a protein in the first influenza virus the same as or orthologous to the viral protein comprised in the second composition in the subject.

In some embodiments, the viral protein of the second influenza virus in the second composition is a recombinant protein, a purified protein, or both.

In some embodiments, the first influenza virus and the second influenza virus are the same.

In some embodiments, the first influenza virus and the second influenza virus are different.

In some embodiments, the first influenza virus and the second influenza viruses are of the same subtype but different strains.

In some embodiments, the first influenza virus and the second influenza viruses are of different subtypes.

In some embodiments, the second composition does not comprise an adjuvant.

In some embodiments, the second composition comprises a pharmaceutically acceptable carrier suitable for intranasal, inhalational, intratracheal, intrapulmonary, and/or intrabronchial administration.

In some embodiments, the administration of the first composition and the second composition results in expansion of antigen-specific, tissue-resident memory T and/or B cell response(s) against the virus in the lung, the airway or the mediastinal lymph nodes (mLNs) of the subject.

In some embodiments, the subject is a mammal.

In some embodiments, the subject is a human.

In some aspects, the present invention is directed to a method of boosting an existing immunity against an influenza viral infection in a subject. In some embodiments, the existing immunity is acquired via a parenteral administration to the subject a first composition comprising a first compound in an amount sufficient to elicit systemic T and/or B cell response(s) against a first influenza virus in the subject.

In some embodiments, the method comprises administering to the subject a second composition comprising a viral protein of an influenza virus through an administration route comprising intranasal, inhalational, intratracheal, intrapulmonary, and intrabronchial, in an amount sufficient to result in establishment of tissue-resident T and/or B cell(s), mucosal IgA, and/or systemic IgG specific against the influenza viral infection in the subject.

In some embodiments, the method further comprises parentally administering to the subject a third composition for boosting the immune response elicited by the first composition or the immune response elicited by both the first composition and the second composition.

In some embodiments, the third composition is administered before or after the second composition.

In some embodiments, the third composition is the same as the first composition.

In some embodiments, the first influenza virus or the second influenza virus is independently an influenza A virus, an influenza B virus, an influenza C virus, or combinations thereof.

In some embodiments, a hemagglutinin protein of the first influenza virus or the second influenza virus is independently an H1 subtype, an H2 subtype, an H3 subtype, an H4 subtype, an H5 subtype, an H6 subtype, an H7 subtype, an H8 subtype, an H9 subtype, an H10 subtype, an H11 subtype, an H12 subtype, an H13 subtype, an H14 subtype, an H15 subtype, an H16 subtype, an H17 subtype, an H18 subtype, or combinations thereof.

In some embodiments, a neuraminidase protein of the first influenza virus or the second influenza virus is independently an N1 subtype, an N2 subtype, an N3 subtype, an N4 subtype, an N5 subtype, an N6 subtype, an N7 subtype, an N8 subtype, an N8 subtype, an N9 subtype, an N10 subtype, an N11 subtype, or combinations thereof.

In some embodiments, the first composition comprises a vaccine against the first influenza virus.

In some embodiments, the vaccine against the first influenza virus comprises at least one selected from the group consisting of a mRNA-lipid nanoparticle (LNP)-based vaccine against the influenza viral infection, a viral vector vaccine against the influenza viral infection, an inactivated virus vaccine against the influenza viral infection, a viral subunit/peptide vaccine against the influenza viral infection, a virus like particle (VLP)-based vaccine against the influenza viral infection, and a DNA vaccine against the influenza viral infection.

In some embodiments, the second composition comprises a surface protein of the second influenza virus.

In some embodiments, the second composition comprises a hemagglutinin protein of the second influenza virus, a neuraminidase protein of the second influenza virus, or combinations thereof.

In some embodiments, the first composition elicits systemic T and/or B cell response(s) against a protein in the first influenza virus the same as or orthologous to the viral protein comprised in the second composition in the subject.

In some embodiments, the viral protein of the second influenza virus in the second composition is a recombinant protein, a purified protein, or both.

In some embodiments, the first influenza virus and the second influenza virus are the same.

In some embodiments, the first influenza virus and the second influenza virus are different.

In some embodiments, the first influenza virus and the second influenza viruses are of the same subtype but different strains.

In some embodiments, the first influenza virus and the second influenza viruses are of different subtypes.

In some embodiments, the second composition does not comprise an adjuvant.

In some embodiments, the second composition comprises a pharmaceutically acceptable carrier suitable for intranasal, inhalational, intratracheal, intrapulmonary, and/or intrabronchial administration.

In some embodiments, the administration of the first composition, the second composition and/or the third composition are separated from each other by about 3 days to about 60 days.

In some embodiments, the method results in expansion of antigen-specific, tissue-resident memory T and/or B cell response(s) against the virus in the lung, the airway or the mediastinal lymph nodes (mLNs) of the subject.

In some embodiments, the subject is a mammal, such as a human.

In some aspects, the present invention is directed to a composition for boosting an existing immunity against an influenza viral infection in a subject.

In some embodiments, the composition comprises a viral protein of an influenza virus.

In some embodiments, the composition further comprises a pharmaceutically acceptable carrier suitable for intranasal, inhalational, intratracheal, intrapulmonary, and/or intrabronchial administration of the protein of the influenza virus.

In some embodiments, the influenza virus is an influenza A virus, an influenza B virus, an influenza C virus, or combinations thereof.

In some embodiments, a hemagglutinin protein of the influenza virus is an H1 subtype, an H2 subtype, an H3 subtype, an H4 subtype, an H5 subtype, an H6 subtype, an H7 subtype, an H8 subtype, an H9 subtype, an H10 subtype, an H11 subtype, an H12 subtype, an H13 subtype, an H14 subtype, an H15 subtype, an H16 subtype, an H17 subtype, an H18 subtype, or combinations thereof.

In some embodiments, a neuraminidase protein of the influenza virus is an N1 subtype, an N2 subtype, an N3 subtype, an N4 subtype, an N5 subtype, an N6 subtype, an N7 subtype, an N8 subtype, an N8 subtype, an N9 subtype, an N10 subtype, an N11 subtype, or combinations thereof.

In some embodiments, the viral protein is a surface protein of the influenza virus.

In some embodiments, the viral protein is a hemagglutinin protein of the influenza virus, a neuraminidase protein of the influenza virus, or combinations thereof.

In some embodiments, the viral protein is a recombinant protein, a purified protein, or both.

In some embodiments, the composition does not comprise an adjuvant.

In some embodiments, the composition further comprising a pharmaceutically acceptable carrier suitable for intranasal, inhalational, intratracheal, intrapulmonary, and/or intrabronchial administration.

In some embodiments, the existing immunity against the influenza viral infection in the subject was acquired response to a parenterally administered influenza vaccination against influenza viral infection.

In some embodiments, the subject is a mammal.

In some embodiments, the subject is a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood. however, that the instant specification is not limited to the precise arrangements and instrumentalities of the embodiments shown in the drawings.

FIG. 1 illustrates certain aspects of a “prime and boost” immunization strategy against influenza A virus, as well as the evaluation of this strategy, in accordance with some embodiments. As shown in FIG. 1, at day 0, BALB/c mice were administered intramuscularly (“i.m.”) with 5 μg of a non-limiting lipid nanoparticle (“LNP”) based influenza vaccine, which contains mRNA molecules encoding the hemagglutinin (HA) protein of the influenza virus stain PR8 (a strain of the H1N1 subtype) (the “prime” step). At either day 7 or 14, the mice were administered intranasally (“i.n.”) with 5 μg of a recombinant HA protein (the “boost” step). Two weeks from the boost step, resident memory T-cells (“T_{RM}”) and resident memory B-cells (“B_{RM}”) were analyzed in the samples of the lung, the mediastinal lymph nodes (mLN), and the bronchoalveolar lavage fluid (BALF) and antibodies were analyzed in the samples of the serum and the BALF.

FIG. 2 demonstrates that the non-limiting “prime and boost” immunization strategy illustrated in FIG. 1 generated robust antigen-specific CD8 T cell responses, in accordance with some embodiments. Comparing with both naïve mice that were not immunized and mice that only received the “priming” LNP based vaccine, the mice that received both the intramuscular “prime” immunization and the intranasal recombinant HA “boost” at day 14 had significantly higher counts of activated resident memory CD8 T cells in the lung (left panel). The mice that received both the intramuscular “prime” immunization and the intranasal recombinant HA “boost” at day 7 had significantly higher counts of activated resident memory CD8 T cells in the lung (left panel) and the BALF in comparison to naïve mice and “prime only” mice. In FIG. 2, “Tet⁺” indicates that the CD8 T cell is reactive with a tetrameric class I MHC protein in complex with HA peptide. “CD44⁺” means that the cell is CD44 positive, which indicates that the CD8 T cell is activated. “IV⁻” indicates that the cell is in the tissue rather than vascular in nature (the animals were injected intravenously (IV) with anti-CD45 labeling antibodies to distinguish circulating from tissue-resident T cell responses).

FIG. 3 demonstrates that the non-limiting “prime and boost” immunization strategy illustrated in FIG. 1 generated robust polyclonal CD4 T cell responses, in accordance with some embodiments. Comparing with both naïve mice that were not immunized and mice that only received the “priming” LNP based vaccine, the mice that received both the intramuscular “prime” immunization and the intranasal recombinant HA “boost” had significantly higher counts of activated, antigen-experienced CD4 T cells in the lung (left panel), the mLN (middle panel) and the BALF (right panel). In FIG. 3, “CD44⁺” means that the cell is CD44 positive, which indicates that the CD4 T cell is activated. “CD69⁺” means that the cell is CD69 positive, which indicates that the CD4 T cell is an antigen-experienced CD4 T cell. “IV⁻” indicates that the cell is in the tissue rather than vascular in nature (the animals were injected intravenously (IV) with anti-CD45 labeling antibodies to distinguish circulating from tissue-resident T cell responses).

FIG. 4 demonstrates that the non-limiting “prime and boost” immunization strategy illustrated in FIG. 1 generated high numbers of IgA-producing B cells, in accordance with some embodiments. Comparing with both naïve mice that were not immunized and mice that only received the LNP based “priming” vaccination, the mice that received both the intramuscular “prime” immunization and the intranasal recombinant HA “boost” had higher counts of IgA-producing resident memory B cells in the lung (left panel), the mLN (middle panel) and the BALF (right panel). In FIG. 4, “IgA⁺” indicates that the cell produces polyclonal IgA. “CD38⁺” means that the cell is CD38 positive, which indicates that the B cell is memory B cell-like rather than antibody secreting plasma cell. “IV” indicates that the cell is in the tissue rather than vascular in nature (the animals were injected intravenously (IV) with anti-CD45 labeling antibodies to distinguish circulating from tissue-resident T cell responses).

FIG. 5 demonstrates that non-limiting “prime and boost” immunization strategy illustrated in FIG. 1 induced robust mucosal immunoglobulin responses. Comparing with both naïve mice that were not immunized and mice that only received the LNP based “priming” immunization, the mice that received both the intramuscular “prime” immunization and the intranasal recombinant HA “boost” had higher concentrations of anti-HA IgA and IgG in the BALF (left and middle panels), and high concentrations of anti-HA IgG in the serum (right panel).

FIG. 6 illustrates certain aspects of a “prime and boost” immunization strategy against influenza A virus, as well as methods of evaluating this strategy, in accordance with some embodiments. As shown in FIG. 6, at day 0), BALB/c mice were administered intramuscularly (“i.m.”) with 1 μg (rather than 5 μg used in the experiments described in FIGS. 1-5) of a non-limiting lipid nanoparticle (LNP) based influenza vaccine, which contains mRNA encoding the HA protein of the influenza virus stain PR8 (the “prime” step). At day 14, the animals were administered intranasally (“i.n.”) with 5 μg of a recombinant HA protein (the “boost” step). Two weeks from the boost step, resident memory T-cells (“T_{RM}”) and resident memory B-cells (“B_{RM}”) were analyzed in the samples of the lung, the mLN, and the BALF, and antibodies were analyzed in the samples of the serum and the BALF.

FIG. 7 demonstrates that the non-limiting “prime and boost” immunization strategy illustrated in FIG. 6 induced robust CD8 T_{RM} and CD4 T_{RM} responses, in accordance with some embodiments. Comparing with both naïve mice that were not immunized and mice that only received the LNP based “priming” immunization, the mice that received both the intramuscular “prime” immunization and the intranasal recombinant HA “boost” had higher counts of activated resident memory CD8 T cells in the lung (leftmost panel) and the BALF (mid-left panel), as well as higher counts of resident memory CD4 T cells in the lung (mid-right panel) and the BALF (rightmost panel). In FIG. 7. “Tet⁺” indicates that the CD8 T cell is reactive with a tetrameric class I MHC protein in complex with HA peptide. “CD44′” means that the cell is CD44 positive, which indicates that the CD8 T or the CD 4 T cell is activated. “IV⁻” indicates that the cell is in the tissue rather than vascular in nature (the animals were injected intravenously (IV) with anti-CD45 labeling antibodies to distinguish circulating from tissue-resident T cell responses). “CD69⁺CD103⁺” means that the CD4 T cell is CD69 positive and CD103 positive, which indicates that the CD4 T cell is a memory CD4 T cell.

FIG. 8 demonstrates that the non-limiting “prime and boost” immunization strategy illustrated in FIG. 6 induces IgA-producing B cells in the lung and the mLN, in accordance with some embodiments. Comparing with both naïve mice that were not immunized and mice that only received the LNP-based “priming” immunization, the mice that received both the intramuscular “prime” immunization and the intranasal recombinant HA “boost” had higher counts of resident memory B cells that express IgA in both the lung (leftmost panel) and the mLN (mid-left panel), and higher antibody secreting cells (ASC) that express IgA in the mLN (rightmost panel). Comparing with mice that only received the “priming” immunization, the mice that received both the intramuscular “prime” immunization and the intranasal recombinant HA “boost” had higher counts of ASCs that express IgA in the mLN (rightmost panel). “IgA'” indicates that the B cell produces polyclonal IgA with unknown specificity. “CD38⁺” means that the cell is CD38 positive, which indicates that the B cell is memory B cell-like rather than antibody secreting cell. “CD138⁺” means that the cell is CD138 positive, which indicates that the cell is an antibody secreting cell. “IV” indicates that the cell is in the tissue rather than vascular in nature (the animals were injected intravenously (IV) with anti-CD45 labeling antibodies to distinguish circulating from tissue-resident T cell responses).

FIG. 9 demonstrates that the non-limiting “prime and boost” immunization strategy illustrated in FIG. 6 induces local mucosal IgA and systemic IgG, in accordance with some embodiments. Comparing with both naïve mice that were not immunized and mice that only received the LNP-based “priming” immunization, the mice that received both the intramuscular “prime” immunization and the intranasal recombinant HA “boost” has significantly higher concentrations of anti-HA IgA in the BALF (left panel), as well as significantly higher concentrations of anti-HA IgG in the BALF (middle panel) and the serum (right panel).

FIG. 10 illustrates certain aspects of a non-limiting “prime and boost” immunization strategy against influenza A virus, as well as the evaluation of this strategy, in accordance with some embodiments. As shown in FIG. 10, at day 0, BALB/c mice were administered intramuscularly (“i.m.”) with 1 μg of a non-limiting lipid nanoparticle (LNP) based influenza vaccine, which contains mRNA encoding the hemagglutinin (HA) protein of the influenza virus stain PR8 (a strain of the H1N1 subtype) (the “prime” step). At day 14, the mice were administered intranasally (“i.n.”) with 1 μg of a recombinant HA protein (the “boost” step). Four weeks from the boost step, resident memory T-cells (“T_{RM}”) and resident memory B-cells (“B_{RM}”) were analyzed in the samples of the lung and the mLN, and antibodies were analyzed in the samples of the serum and the BALF.

FIG. 11 depicts the levels of antigen-specific CD8⁺ resident memory T cells at 4 weeks after the non-limiting “prime and boost” immunizations illustrated in FIG. 10, in accordance with some embodiments. Left panel: counts of tissue resident memory CD8 T cells in the lungs of naïve mice (left), mice primed with the LNP based influenza vaccine and boosted with the same LNP based vaccine (middle), and mice primed with the LNP based influenza vaccine and boosted with the intranasal administered recombinant HA protein (right). Right panel: counts of tissue resident memory CD8 T cells in the mLN of naïve mice (left), mice primed with the LNP based influenza vaccine and boosted with the same LNP based vaccine (middle), and mice primed with the LNP based influenza vaccine and boosted with the intranasal administered recombinant HA protein (right). In FIG. 11, “Tett” indicates that the CD8 T cell is reactive with a tetrameric class I MHC protein in complex with HA peptide. “CD44⁺” means that the cell is CD44 positive, which indicates that the CD8 T cell is activated. “IV⁻” indicates that the cell is in the tissue rather than vascular in nature (the animals were injected intravenously (IV) with anti-CD45 labeling antibodies to distinguish circulating from tissue-resident T cell responses).

FIG. 12 depicts the levels of IgA-producing tissue resident memory B cells at 4 weeks after the non-limiting “prime and boost” immunizations illustrated in FIG. 10, in accordance with some embodiments. Left panel: counts of tissue resident memory B cells that express IgA with unknown specificity in the lungs of naïve mice (left), mice primed with the LNP based influenza vaccine and boosted with the same LNP based vaccine (middle), and mice primed with the LNP based influenza vaccine and boosted with the intranasal administered recombinant HA protein. Right panel: counts of tissue resident memory B cells that express IgA with unknown specificity in the mediastinal lymph nodes (mLN) of naïve mice (left), mice primed with the LNP based influenza vaccine and boosted with the same LNP based vaccine (middle), and mice primed with the LNP based influenza vaccine and boosted with the intranasal administered recombinant HA protein. In FIG. 12, “IgA⁺” means the cell produces IgA with unknown specificity. “IV⁻” indicates that the cell is in the tissue rather than vascular in nature (the animals were injected intravenously (IV) with anti-CD45 labeling antibodies to distinguish circulating from tissue-resident T cell responses).

FIG. 13 demonstrates that the non-limiting “prime and boost” immunizations illustrated in FIG. 10 induced robust local and systemic antibody responses, in accordance with some embodiments. Specifically, in comparison with naïve mice that did not receive immunization (“Naive”) or mice that received the LNP based influenza vaccine twice (“Prime+Boost”), mice that received the non-limiting “prime and boost” immunization illustrated in FIG. 10 (“Prime+rHA”) have significantly higher counts of anti-HA IgA in both BALF (left most panel) and serum (mid-left panel). In comparison with mice that received the LNP based influenza vaccine twice (“Prime+Boost”), mice that received the non-limiting immunization illustrated in FIG. 10 (“Prime+rHA”) have comparable levels of anti-HA IgG in the BALF (mid right panel) and the serum (rightmost panel).

FIGS. 14A-14B demonstrate that a non-limiting “prime and boost” immunization strategies using intranasally administered recombinant HA protein as the booster according to some embodiments herein confer better protection against heterologous viral infection than similar immunization strategies using intramuscularly an administered booster. FIG. 14A illustrates certain aspects of a “prime and boost” immunization strategy against influenza A virus, as well as the evaluation of this strategy, in accordance with some embodiments. Specifically, BALB/c mice were administered intramuscularly with a non-limiting lipid nanoparticle (LNP) based influenza vaccine, which contains mRNA encoding the hemagglutinin (HA) protein of the influenza virus stain PR8 (a strain of the H1N1 subtype) (the “prime” step). At day 14, the animals were administered intranasally (“i.n.”) with of a recombinant HA protein (the “boost” step). At day 56, the mice were challenged with either the homologous PR8 virus (10⁴ PFU). The weight loss and survival of the mice were tracked for the next two weeks. FIG. 14B demonstrates that the non-limiting intranasal HA protein booster conferred comparable levels of protection against homologous viral infection-caused weight loss to the LNP based boost, and both of which are significantly better than non-immunization (left panel).

FIGS. 15A-15B demonstrate that the “prime and boost” immunization strategies using intranasally administered recombinant HA protein as the booster according to some embodiments herein confer better protection to heterologous viral infection than similar immunization strategies using intramuscularly administered boost. FIG. 15A illustrates certain aspects of a non-limiting “prime and boost” immunization strategy against influenza A virus. as well as methods of evaluating this strategy, in accordance with some embodiments. Specifically, BALB/c mice were administered intramuscularly with a non-limiting lipid nanoparticle (LNP) based influenza vaccine, which contains mRNA encoding the hemagglutinin (HA) protein of the influenza virus stain PR8 (a strain of the H1N1 subtype) (the “prime” step). At day 14, the animals were administered intranasally (“i.n.”) with of a recombinant HA protein (the “boost” step). At day 54, the mice were challenged with either the homologous PR8 virus or with the heterologous influenza virial strain A/Beijing/262/95, which is of H1N1 subtype. The weight loss and survival of the mice were tracked for the next two weeks. FIG. 15B demonstrated that the non-limiting intranasal HA protein booster conferred substantially better levels of protection against the heterologous viral infection-caused weight loss than the LNP based booster, “prime” only, and non-immunization.

FIG. 16A: Balb/c mice were immunized intramuscularly with 0.05 μg of PR8 HA mRNA-LNP and received the second dose 2 weeks after the initial dose. Three weeks thereafter, mice received the third dose. Six weeks after the third dose, mice were intranasally challenged with 10000PFU of A/PR8 and lungs were collected 3 days after infection. FIG. 16B: Viral RNA was quantified by RT-qPCR using primers specific to nucleoprotein (NP) or HA. FIG. 16B demonstrated that the reduction of viral replication by the “prime and boost” strategy using the intranasal HA protein booster or the LNP based parenteral prime and boost. The LNP based parenteral prime and boost followed by the non-limiting intranasal HA protein booster (designated as Prime+boost+HA in the FIG. 16B), however; resulted in the nearly complete inhibition of viral replication in the lung. Additional intranasal HA protein booster after the “prime and boost” strategy (designated as Prime+HA+HA in the FIG. 16B) conferred significantly better protection.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Currently, most of the clinically approved vaccines for seasonal influenza viruses are inactivated virus-based and administered intramuscularly. While such parenteral vaccines are generally safe and capable of generating systemic immunity by inducing virus-specific IgG in the circulation, their efficacy varies depending on the matching of the vaccine seed virus strains and circulating viruses. One of the reasons why parenteral influenza vaccines are sensitive to the mismatch of the virus strains is because they produce very few T cells and mucosal IgA in the respiratory tract, which target more conserved epitopes and/or are more broadly reactive than circulating IgG.

In addition to the broadly protective properties of mucosal immune responses in the respiratory tract, the respiratory mucosa has the spatial advantage to encounter incoming viruses at the site of infection. Influenza viruses invade the body through the respiratory mucosa and then are later transmitted via respiratory droplets and aerosols. Thus, vaccine strategies that elicit mucosal immune responses can potentially prevent infection and transmission. Such protective mucosal immune responses can be gained by natural infection and/or vaccination through respiratory mucosa. Therefore, intranasal vaccination is a promising platform for broadly protective vaccine development. Currently, a live-attenuated intranasal influenza vaccine (FluMist) is available. However, the efficacy of that vaccine varies from year to year due to the need to match the vaccine to circulating strains and the attenuation process that result in loss of immunogenicity. FluMist approval is limited to 2-49-year-old individuals because of the lack of effectiveness to prevent febrile illness in older people, and the vaccine is not approved for immunocompromised people. Thus, vaccine strategies that aim to elicit potent protective immune effector mechanisms at mucosal surfaces are much needed to provide front-line protection against seasonal influenza viruses in people of all ages.

The study described herein (“the present study.”) developed a novel immunization strategy against influenza infection. According to this strategy, the subject is first administered a parenteral vaccine to elicit systemic T and B cell responses (sometimes referred to as “priming” herein). The subject is then administered a booster, such as a viral surface protein administered through the airway (sometimes referred to as “boosting” herein).

The present study, using mice as a model animal, confirmed that the prime and airway boost immunization strategy resulted in dramatic expansion of antigen-specific. tissue-resident memory T and B cell responses in the lung parenchyma and airways. Furthermore, mice immunized with the prime and airway boost strategy developed much higher levels of airway and systemic IgA and IgG antibodies compared to mice that were primed without the airway boosters. In a mouse model of influenza viral infection, prime and airway boost reduced diseases. Notably, the present study discovered that an adjuvant is not required in the boosting step (although does not have to be excluded) to achieve the desirable effects of robustly boosting the immunity against influenza viral infection.

In another aspect, the production of conventional inactivated virus vaccines and live-attenuated virus vaccines requires culturing live virus, which allows mutations in the viral genome to happen. Viral culturing is often performed using chicken eggs, thus rendering the produced vaccines unusable for people with egg protein allergies. In contrast, the boosters used in the present immunization strategy can be made from recombinant viral proteins, which completely avoids the viral culturing steps.

In another aspect, the present study discovered that the “prime and airway boost” strategy described herein, when combined with the conventional “prime and boost” (i.e., a parental administration of priming shot followed by parental administration(s) of boosting shot), results in dramatically increased immunity against viral infections.

Accordingly, in some aspects, the present invention is directed to a method of treating, ameliorating, and/or preventing an influenza viral infection in a subject in need thereof, and/or immunizing against an influenza viral infection in a subject.

In some aspects, the present invention is directed to a composition for boosting vaccination against an influenza viral infection in a subject.

In some aspects, the present invention is directed to a kit for vaccinating a subject against an influenza viral infection.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “composition” or “pharmaceutical composition” refers to a mixture of at least one compound useful within the specification with a pharmaceutically acceptable carrier. The pharmaceutical composition facilitates administration of the compound to a patient or subject. Multiple techniques of administering a compound exist in the art including, but not limited to, intravenous, subcutaneous, oral, aerosol, parenteral, ophthalmic, nasal, inhalational, intratracheal, intrapulmonary, intrabronchial, and topical administration.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the specification within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the specification, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the specification, and are physiologically acceptable to the patient. Supplementary active compounds may also be incorporated into the compositions. The “pharmaceutically acceptable carrier” may further include a pharmaceutically acceptable salt of the compound useful within the instant specification. Other additional ingredients that may be included in the pharmaceutical compositions used in the practice of the instant specification are known in the art and described, for example in Remington's Pharmaceutical Sciences (Genaro, Ed., Mack Publishing Co., 1985, Easton, PA), which is incorporated herein by reference.

As used herein, the term “prevention” or “preventing” encompasses prophylaxis. Accordingly, the compositions and methods of the instant specification include prophylactic applications. Therefore prevention” of or “preventing” a state, disorder or condition includes preventing or delaying the appearance of clinical symptoms of the state, disorder or condition developing in a subject that may be afflicted with or predisposed to the state, disorder or condition but does not yet experience or display clinical or subclinical symptoms of the state, disorder or condition.

As used herein, the term “prime” refers to immunizing a subject by administering a vaccine parenterally to elicit a systemic T and/or B cell response(s) against a viral infection in the subject. As used herein, the term “boost” refers to administering to the same subject a vaccine, such as via the airway, against the same or related viral infections after the “prime.”

As used herein, “treating a disease or disorder” means reducing the frequency and/or severity with which a symptom of the disease or disorder is experienced by a patient. Disease and disorder are used interchangeably herein.

As used herein, the term “treatment” or “treating” encompasses therapy. Accordingly, the compositions and methods of the instant specification include therapeutic applications. Therefore “treating” or “treatment” of a state, disorder or condition includes: (i) inhibiting the state, disorder or condition, i.e., arresting or reducing the development of the disease or at least one clinical or subclinical symptom thereof, and/or (ii) relieving the disease, i.e., causing regression of the state, disorder or condition or at least one of its clinical or subclinical symptoms.

By the term “vaccine” as used herein, is meant any compositions, viruses, proteins, nucleic acids, or the like, which serve to protect a subject against a disease caused by an influenza virus and/or to treat a subject already infected with an influenza virus compared with an otherwise identical subject to which the vaccine is not administered or compared with the animal prior to the administration of the vaccine, by specifically increasing the subject's immunity against the influenza virus.

Ranges: throughout this disclosure, various aspects can be presented in a range format. It should be understood that the description in range format is merely for convenience and brevity and should not be construed as an inflexible limitation on the scope of the instant specification. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Abbreviations: BALF: bronchoalveolar lavage fluid. B_{RM}: resident memory B cell. HA: hemagglutinin. LNP: lipid nanoparticle, mLN: mediastinal lymph node. NA: neuraminidase. PR8: influenza strain A/Puerto Rico/8/1934 (H1N1 subtype). T_{RM}: resident memory T cell.

### Method of Treating, Ameliorating, and/or Preventing Influenza Viral Infection, and/or Method of Immunizing against Influenza Viral Infection

In some aspects, the present study developed a novel immunization strategy against influenza viral infection, which includes administering parentally an influenza vaccine (“prime”) to a subject, and then administering an influenza viral surface protein in the airway of the subject (“boost”). The present study confirmed, using a mouse model, that immunizing a subject according to the prime and boost strategy resulted in dramatic expansion of antigen-specific, tissue-resident memory T and B cell responses in the lung parenchyma and airways, and much higher levels of airway and systemic IgA and IgG antibodies compared to subjects that were primed without the airway boosting. The present study further confirmed, using a mouse model of influenza viral infection, the prime and airway boost immunization strategy reduced the severity of diseases.

Notably, the present study discovered that in certain non-limiting embodiments the influenza viral surface protein administered in the airway does not require adjuvants for the observed robust immunity to develop. The present study further discovered that the immunization strategy is able to generate broad-spectrum immunity effective against influenza subtypes or strains different from the subtypes/strains used to generate the immunity.

Accordingly, in some aspects, the present invention is directed to a method of treating, ameliorating and/or preventing an influenza viral infection in a subject in need thereof.

In some aspects, the present invention is directed to a method of immunizing a subject against influenza viral infection.

In some embodiments, the method includes:
parenterally administering to the subject a first composition comprising a first compound in an amount sufficient to elicit systemic T and/or B cell response(s) against the first influenza virus in the subject; and
administering to the subject a second composition comprising a viral protein of a second influenza virus through an administration route comprising intranasal, inhalational, intratracheal, intrapulmonary, and intrabronchial, in an amount sufficient to result in establishment of a tissue-resident T and/or B cell(s) or mucosal IgA specific against infection of the first influenza virus in the subject.

In some embodiments, the first influenza virus or the second influenza virus is independently an influenza A virus, an influenza B virus, an influenza C virus, or combinations thereof.

In some embodiments, a hemagglutinin protein of the first influenza virus or the second influenza virus is independently an H1 subtype, an H2 subtype, an H3 subtype, an H4 subtype, an H5 subtype, an H6 subtype, an H7 subtype, an H8 subtype, an H9 subtype, an H10 subtype, an H11 subtype, an H12 subtype, an H13 subtype, an H14 subtype, an H15 subtype, an H16 subtype, an H17 subtype, an H18 subtype, or combinations thereof.

In some embodiments, a neuraminidase protein of the first influenza virus or the second influenza virus is independently an N1 subtype, an N2 subtype, an N3 subtype, an N4 subtype, an N5 subtype, an N6 subtype, an N7 subtype, an N8 subtype, an N8 subtype, an N9 subtype, an N10 subtype, an N11 subtype, or combinations thereof.

In some embodiments, the first composition comprises a vaccine against the first influenza virus.

In some embodiments, the vaccine against the first influenza virus comprises at least one selected from the group consisting of a mRNA-lipid nanoparticle (LNP)-based vaccine against the influenza viral infection, a viral vector vaccine against the influenza viral infection, an inactivated virus vaccine against the influenza viral infection, a viral subunit/peptide vaccine against the influenza viral infection, a virus like particle (VLP)-based vaccine against the influenza viral infection, and a DNA vaccine against the influenza viral infection.

In some embodiments, the second composition comprises a surface protein of the second influenza virus.

In some embodiments, the second composition comprises a hemagglutinin protein of the second influenza virus, a neuraminidase protein of the second influenza virus, or combinations thereof.

In some embodiments, the first composition elicits systemic T and/or B cell response(s) against a protein in the first influenza virus the same as or orthologous to the viral protein comprised in the second composition in the subject.

In some embodiments, the viral protein of the second influenza virus in the second composition is a recombinant protein, a purified protein, or both.

In some embodiments, the first influenza virus and the second influenza virus are the same.

In some embodiments, the first influenza virus and the second influenza virus are different. In some embodiments, the first influenza virus and the second influenza viruses are of the same subtype but different strains. In some embodiments, the first influenza virus and the second influenza viruses are of different subtypes.

In some embodiments, the second composition does not comprise an adjuvant.

In some embodiments, the second composition comprises a pharmaceutically acceptable carrier suitable for intranasal, inhalational, intratracheal, intrapulmonary, and/or intrabronchial administration of the second composition.

In some embodiments, the administration of the first composition and the administration of the second composition are separated by about 3 days to about 60 days, such as by about 4 days to about 30 days, about 5 days to about 20 days, or about 7 days to about 14 days. In some embodiments, the administration of the first composition and the administration of the second composition are separated by about 3 days, about 5 days, about 7 days, about 10 days, about 14 days, about 15 days, about 21 days, about 28 days, about 30 days, or any ranges therebetween.

In some embodiments, the method results in expansion of antigen-specific, tissue-resident memory T and/or B cell response(s) against the virus in the lung, the airway or the mediastinal lymph nodes (mLNs) of the subject.

In some embodiments, the subject is a mammal, such as a human.

In some embodiments, the subject is administered parenterally with a third composition.

In some embodiments, the third composition is administered between the administrations of the first and the second compositions. In some embodiments, the third composition boots the immunity elicited by the first composition in the subject.

In some embodiments, the third composition is administered after the administration of the first and the second composition. In some embodiments, the third composition boosts the immunity elicited by both the first composition and the second composition in the subject.

In some embodiments, the third composition is the same as the first composition. In some embodiments, the third composition is a composition according to the description of the first composition herein, but is different from the first composition actually administered in the subject.

### Kit for Treating, Ameliorating, and/or Preventing Influenza Viral Infection, and/or Kit for Immunizing against Influenza Viral Infection

In some aspects, the present study developed a two-composition kit for an immunization strategy against influenza viral infection, which includes a first parentally administered influenza vaccine composition (composition for “priming”), and a second composition including an influenza viral surface protein for administering in the airway (composition for “boosting”). The present study confirmed, using a mouse model, that immunizing a subject with the first “priming” composition and then with the second “boosting” composition resulted in dramatic expansion of antigen-specific, tissue-resident memory T and B cell responses in the lung parenchyma and airways, and much higher levels of airway and systemic IgA and IgG antibodies compared to subjects that were immunized with only the conventional parentally administered influenza vaccine. The present study further confirmed, using a mouse model of influenza viral infection, the administration of the two-composition immunization kit reduced the severity of diseases.

Notably, the present study discovered that the influenza viral protein of the second composition not require adjuvants for the observed robust immunity to develop. The present study further discovered that the administration of the two compositions can generate broad-spectrum immunity effective against influenza subtypes or strains different from the subtypes/strains used in the compositions.

Accordingly, in some aspects, the present invention is directed to a kit for treating, ameliorating, and/or preventing an influenza viral infection in a subject in need thereof.

In some aspects, the present invention is directed to a kit for immunizing a subject against an influenza viral infection.

In some embodiments, the kit includes a first composition and a second composition (and optionally the third composition), which are the same as or similar to those described elsewhere herein, such as in the “Method of Treating, Ameliorating, and/or Preventing Influenza Viral Infection, and/or Method of Immunizing against Influenza Viral Infection” section.

### Method of Boosting Immunity Against Influenza Viral Infection

In some aspects, the present study developed a novel method of boosting an existing immunity against influenza viral infection in a subject, which was generated by administering parentally an influenza vaccine (i.e., the immune system of the subject has been “primed” against influenza viral infection). The present study discovered that, by administering an influenza viral surface protein in the airway of the “primed” subject (i.e., “boosting” the subject), strong immunity against the influenza viral infection can be developed. The present study confirmed, using a mouse model, that “boosting” the subject resulted in dramatic expansion of antigen-specific, tissue-resident memory T and B cell responses in the lung parenchyma and airways, and much higher levels of airway and systemic IgA and IgG antibodies compared to subjects that were primed but did not receive the airway boosting. The present study further confirmed, using a mouse model of influenza viral infection, the airway boost method reduced the severity of diseases.

Notably, the present study discovered that the influenza viral surface protein administered in the airway does not require any adjuvants for the observed robust immunity to develop. The present study further discovered that the boosted immunity is a broad-spectrum immunity effective against influenza subtypes or strains different from the subtypes/strains used to generate the immunity.

Accordingly, in some aspects, the present invention is directed to a method of boosting an existing immunity against an influenza viral infection in a subject. In some embodiments, the existing immunity is acquired using the method and/or composition described elsewhere herein, such as in the “Method of Treating, Ameliorating, and/or Preventing Influenza Viral Infection, and/or Method of Immunizing against Influenza Viral Infection” and “Kit for Treating, Ameliorating, and/or Preventing Influenza Viral Infection, and/or Kit for Immunizing against Influenza Viral Infection” sections.

In some embodiments, the method includes administering to the subject a composition comprising a viral protein of an influenza virus through an administration route comprising intranasal, inhalational, intratracheal, intrapulmonary, and intrabronchial, in an amount sufficient to result in establishment of tissue-resident T and/or B cell(s), mucosal IgA, and/or systemic IgG specific against the influenza viral infection in the subject.

In some embodiments, the composition, as well as the method of administration, is the same as or similar to those described elsewhere, such as in the “Method of Treating, Ameliorating, and/or Preventing Influenza Viral Infection, and/or Method of Immunizing against Influenza Viral Infection” and “Kit for Treating, Ameliorating, and/or Preventing Influenza Viral Infection, and/or Kit for Immunizing against Influenza Viral Infection” sections.

### Composition for Boosting Immunity Against Influenza Viral Infection

In some aspects, the present study developed a novel composition for boosting an existing immunity against influenza viral infection in a subject, which has previously received a parentally administered influenza (i.e., the immune system of the subject has been “primed” against influenza viral infection previously). The present study discovered that, by administering a composition including an influenza viral surface protein in the airway of the “primed” subject (i.e., “boosting” the subject), strong immunity against the influenza viral infection can be developed. The present study confirmed, using a mouse model, that “boosting” the subject with the composition resulted in dramatic expansion of antigen-specific, tissue-resident memory T and B cell responses in the lung parenchyma and airways, and much higher levels of airway and systemic IgA and IgG antibodies compared to subjects that were primed but did not receive the airway boosting composition. The present study further confirmed, using a mouse model of influenza viral infection, the administration of the composition for airway boost reduced the severity of diseases.

Notably, the present study discovered that the boosting composition does not require adjuvants for the observed robust immunity to develop. The present study further discovered that the boosted immunity is a broad-spectrum immunity effective against influenza subtypes or strains different from the subtypes/strains used to generate the immunity.

Accordingly, in some embodiments, the present invention is directed to a composition for boosting an existing immunity against an influenza viral infection in a subject.

In some embodiments, the composition, as well as the method of using the composition, is the same as or similar to those as detailed elsewhere herein, such as in the “Method of Treating. Ameliorating, and/or Preventing Influenza Viral Infection, and/or Method of Immunizing against Influenza Viral Infection” and “Kit for Treating, Ameliorating, and/or Preventing Influenza Viral Infection, and/or Kit for Immunizing against Influenza Viral Infection” sections.

### Combination Therapies

The compounds and/or methods of the present invention are intended to be useful in the methods of present invention in combination with one or more additional compounds useful for treating the diseases or disorders contemplated within the invention. These additional compounds may comprise compounds of the present invention or compounds, e.g., commercially available compounds, known to treat, prevent, or reduce the symptoms of the diseases or disorders contemplated within the invention. Non-limiting examples of compounds that treat, prevent, or reduce the symptoms of influenza viral infection include decongestants such as pseudoephedrine; cough suppressants such as dextromethorphan or guaifenesin; nonsteroidal anti-inflammatory drug (NSAIDs) such as aspirin or ibuprofen; analgesics such as paracetamol (acetaminophen); antiviral drugs such as peramivir. zanamivir, or oseltamivir; and the like.

A synergistic effect may be calculated, for example, using suitable methods such as, for example, the Sigmoid-Eₘₐₓ equation (Holford & Scheiner, 19981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosing

In clinical settings, delivery systems for the compositions described herein can be introduced into a subject by any of a number of methods, each of which is familiar in the art. For instance, a pharmaceutical formulation of the composition can be administered by inhalation or systemically, e.g., by intravenous injection.

The regimen of administration may affect what constitutes an effective amount. The therapeutic formulations may be administered to the subject either prior to or after the manifestation of symptoms associated with the disease or condition. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the composition of the instant specification to a subject, preferably a mammal, more preferably a human, may be carried out using known procedures, at dosages and for periods of time effective to treat a disease or condition in the subject. An effective amount of the composition necessary to achieve a therapeutic effect may vary according to factors such as the time of administration; the duration of administration; other drugs, compounds or materials used in combination with the composition; the state of the disease or disorder; age, sex, weight, condition, general health and prior medical history of the subject being treated; and like factors well-known in the medical arts. Dosage regimens may be adjusted to provide the optimum therapeutic response. For example, several divided doses may be administered daily or the dose may be proportionally reduced as indicated by the exigencies of the therapeutic situation. One of ordinary skill in the art would be able to study the relevant factors and make the determination regarding the effective amount of the composition without undue experimentation. Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the instant specification include oral, nasal, rectal, intravaginal, parenteral, buccal, sublingual or topical. The compounds for use in the instant specification may be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the instant specification are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the instant specification may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type. Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds of the instant specification may be formulated for injection or infusion, for example, intravenous, intramuscular, or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Buccal, Pulmonary, Inhalational, Intranasal Administration, and So Forth

A pharmaceutical composition of the invention may be prepared, packaged, or sold in a formulation suitable for pulmonary administration via the buccal cavity. Such a formulation may comprise dry particles that comprise the active ingredient and have a diameter in the range from about 0.5 to about 7 micrometers, and in certain embodiments from about 1 to about 6 micrometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In certain embodiments, such powders comprise particles wherein at least 98% of the particles by weight have a diameter greater than 0.5 micrometers and at least 95% of the particles by number have a diameter less than 7 micrometers. In certain embodiments, at least 95% of the particles by weight have a diameter greater than 1 micrometer and at least 90% of the particles by number have a diameter less than 6 micrometers. Dry powder compositions may include a solid fine powder diluent such as sugar and are conveniently provided in a unit dose form.

Low boiling propellants generally include liquid propellants having a boiling point of below 65 oF at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in certain embodiments having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration in certain embodiments have an average diameter in the range from about 0.1 to about 200 micrometers.

The pharmaceutical composition of the invention may be delivered using an inhalator such as those recited in U.S. Pat. No. 8,333,192 B2, which is incorporated herein by reference in its entirety.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patents Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the instant specification may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the instant specification may be administered in the form of microparticles, for example, by injection or in the form of wafers or discs by implantation.

In certain embodiments, the compounds of the instant specification are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the instant specification depends on the age, sex and weight of the patient, the current medical condition of the patient and the progression of a disease or disorder contemplated herein in the patient being treated. The skilled artisan is able to determine appropriate dosages depending on these and other factors.

A suitable dose of a compound of the instant specification may be in the range of from about 0.001 mg to about 5.000 mg per day, such as from about 0.01 mg to about 1.000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

Actual dosage levels of the cells in the pharmaceutical formulations of the instant specification may be varied so as to obtain an amount of the composition that are effective to achieve the desired therapeutic response for a particular subject, composition, and mode of administration, without being toxic to the subject.

Toxicity and therapeutic efficacy of such therapeutic regimens are optionally determined in cell cultures or experimental animals, including, but not limited to, the determination of the LD₅₀ (the dose lethal to 50% of the population) and the ED50 (the dose therapeutically effective in 50% of the population). The dose ratio between the toxic and therapeutic effects is the therapeutic index, which is expressed as the ratio between LD50 and ED_{50.} The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1

### “Prime and Airway Boost” Immunization Induced Robust T Cell Response and Mucosal IgA Response

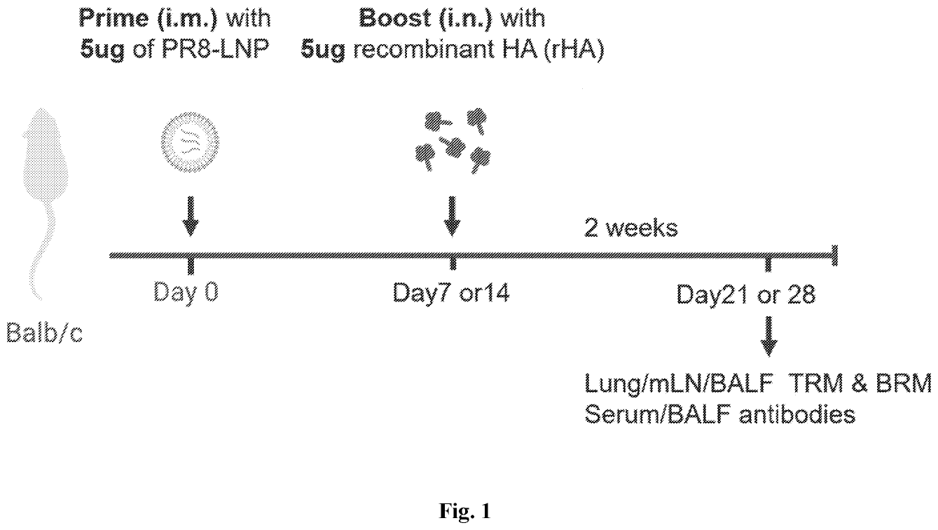

Referring to FIG. 1, at day 0, BALB/c mice were administered intramuscularly (“i.m.”) with 5 μg of a non-limiting lipid nanoparticle (“LNP”) based influenza vaccine, which contains mRNA molecules encoding the hemagglutinin (HA) protein of the influenza virus stain PR8 (a strain of the H1N1 subtype) (the “prime” step). At either day 7 or 14, the mice were administered intranasally (“i.n.”) with 5 μg of a recombinant HA protein of the influenza virus stain PR8 (the “boost” step). Two weeks from the boost step, resident memory T-cells (“T_{RM}”) and resident memory B-cells (“B_{RM}”) were analyzed in the samples of the lung, the mediastinallymph nodes (mLN), and the bronchoalveolar lavage fluid (BALF) and antibodies were analyzed in the samples of the serum and the BALF.

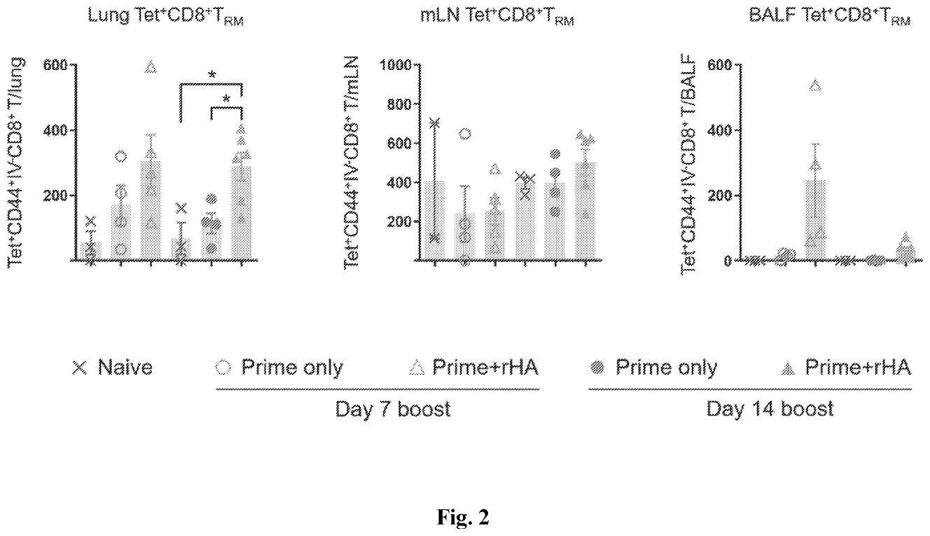

Referring to FIG. 2, the non-limiting “prime and airway boost” immunization strategy generated robust antigen-specific CD8 T cell responses. Specifically, comparing with both naïve mice that were not immunized and mice that only received the “priming” LNP based vaccine, the mice that received both the intramuscular “prime” immunization and the intranasal recombinant HA “airway boost” at day 14 had significantly higher counts of activated resident memory CD8 T cells in the lung (left panel). The mice that received both the intramuscular “prime” immunization and the intranasal recombinant HA “boost” at day 7 had significantly higher counts of activated resident memory CD8 T cells in the lung (left panel) and the BALF in comparison to naïve mice and “prime only” mice.

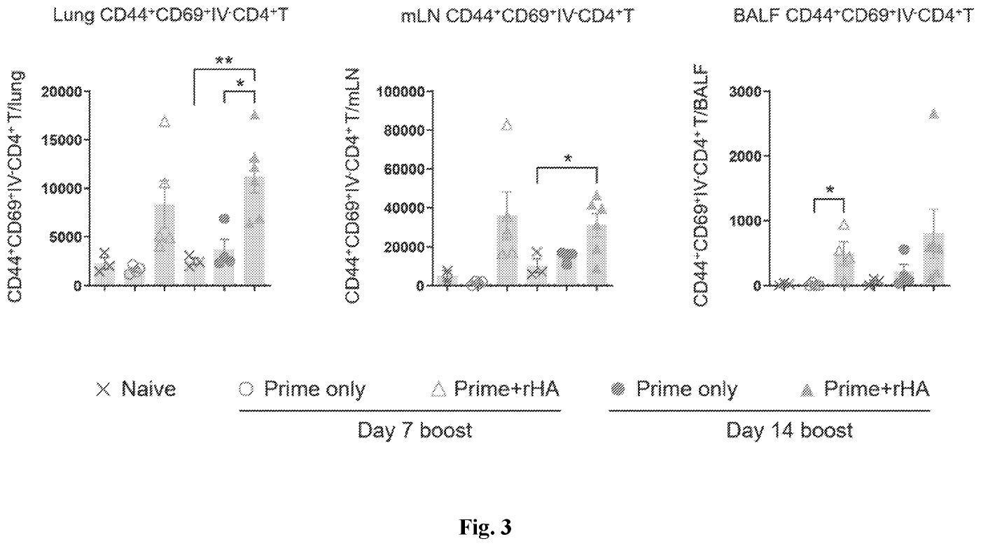

Referring to FIG. 3, the non-limiting “prime and airway boost” immunization strategy generated robust CD4 T cell responses, in accordance with some embodiments. Comparing with both naïve mice that were not immunized and mice that only received the “priming” LNP based vaccine, the mice that received both the intramuscular “prime” immunization and the intranasal recombinant HA “boost” had significantly higher counts of activated, antigen-experienced CD4 T cells in the lung (left panel), the mLN (middle panel) and the BALF (right panel).

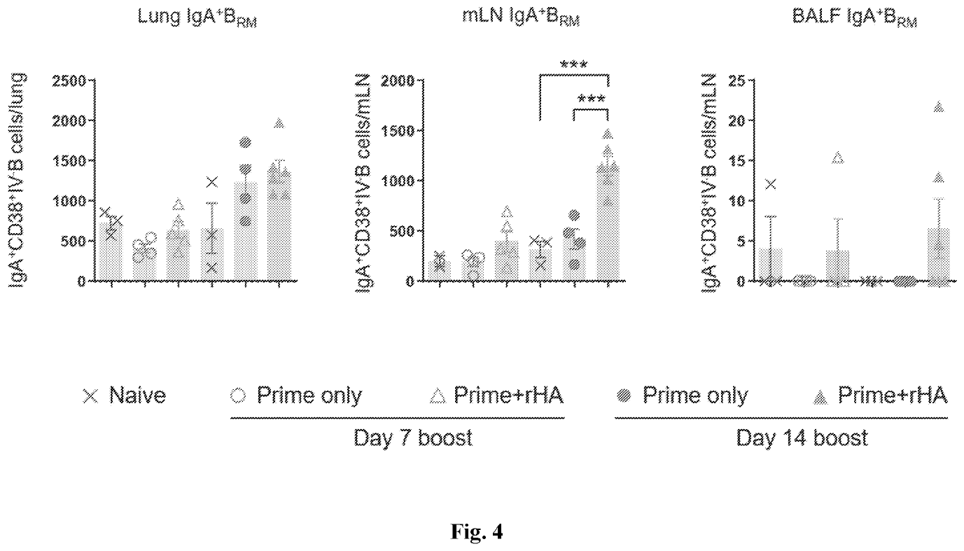

Referring to FIG. 4, the non-limiting “prime and airway boost” immunization strategy generated high numbers of IgA-producing B cells. Specifically, comparing with both naïve mice that were not immunized and mice that only received the LNP based “priming” vaccination, the mice that received both the intramuscular “prime” immunization and the intranasal recombinant HA “boost” had higher counts of IgA-producing resident memory B cells in the lung (left panel), the mLN (middle panel) and the BALF (right panel).

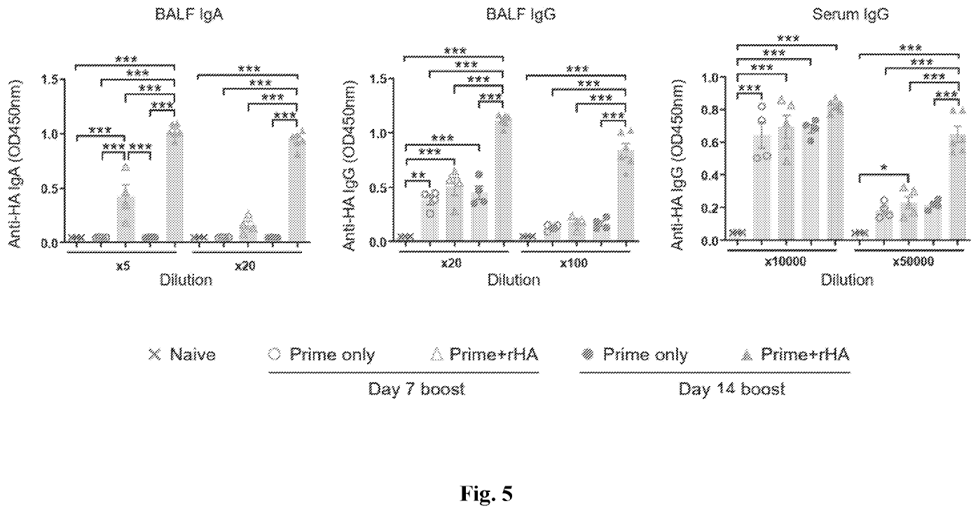

Referring to FIG. 5, the non-limiting “prime and airway boost” immunization strategy induced robust mucosal immunoglobulin responses. Comparing with both naïve mice that were not immunized and mice that only received the LNP based “priming” immunization, the mice that received both the intramuscular “prime” immunization and the intranasal recombinant HA “boost” had higher concentrations of anti-HA IgA and IgG in the BALF (left and middle panels), and high concentrations of anti-HA IgG in the serum (right panel).

### Example 2

### “Prime and Airway Boost” Immunization is Effective With Low Priming Dosage

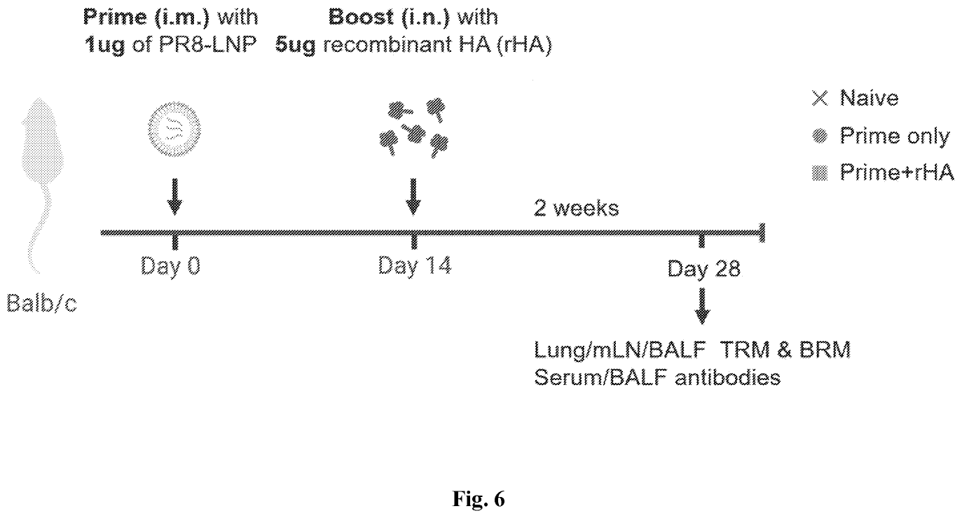

Referring to FIG. 6, at day 0, BALB/c mice were administered intramuscularly (“i.m.”) with 1 μg (rather than 5 μg used in the experiments described in Example 1 herein) of a non-limiting lipid nanoparticle (LNP) based influenza vaccine, which contains mRNA encoding the HA protein of the influenza virus stain PR8 (the “prime” step). At day 14, the animals were administered intranasally (“i.n.”) with 5 μg of a recombinant HA protein of the influenza virus stain PR8 (the “boost” step). Two weeks from the boost step, resident memory T-cells (“T_{RM}”) and resident memory B-cells (“B_{RM}”) were analyzed in the samples of the lung, the mLN, and the BALF, and antibodies were analyzed in the samples of the serum and the BALF.

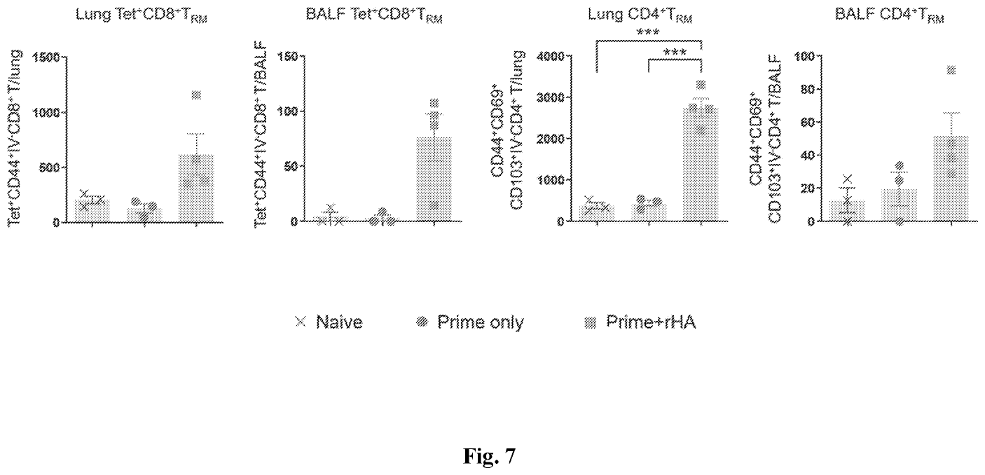

Referring to FIG. 7, the non-limiting “prime and airway boost” immunization strategy induced robust CD8 T_{RM} and CD4 T_{RM} responses. Comparing with both naïve mice that were not immunized and mice that only received the LNP based “priming” immunization, the mice that received both the intramuscular “prime” immunization and the intranasal recombinant HA “boost” had higher counts of activated resident memory CD8 T cells in the lung (leftmost panel) and the BALF (mid-left panel), as well as higher counts of resident memory CD4 T cells in the lung (mid-right panel) and the BALF (rightmost pandel).

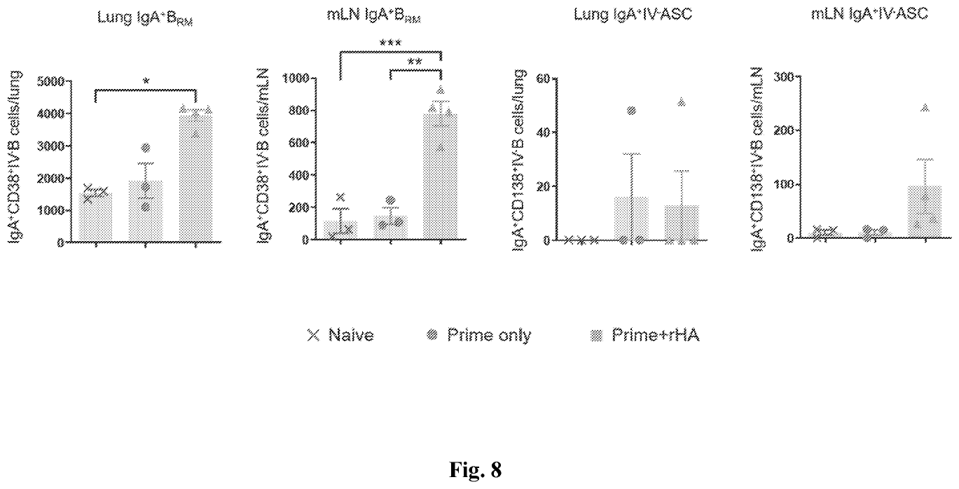

Referring to FIG. 8, the non-limiting “prime and airway boost” immunization strategy induced IgA-producing B cells in the lung and the mLN. Comparing with both naïve mice that were not immunized and mice that only received the LNP-based “priming” immunization, the mice that received both the intramuscular “prime” immunization and the intranasal recombinant HA “boost” had higher counts of resident memory B cells that express IgA in both the lung (leftmost panel) and the mLN (mid-left panel), and higher antibody secreting cells (ASC) that express anti-HA IgA in the mLN (rightmost panel). Comparing with mice that only received the “priming” immunization, the mice that received both the intramuscular “prime” immunization and the intranasal recombinant HA “boost” had higher counts of ASCs that express IgA in the mLN (rightmost panel).

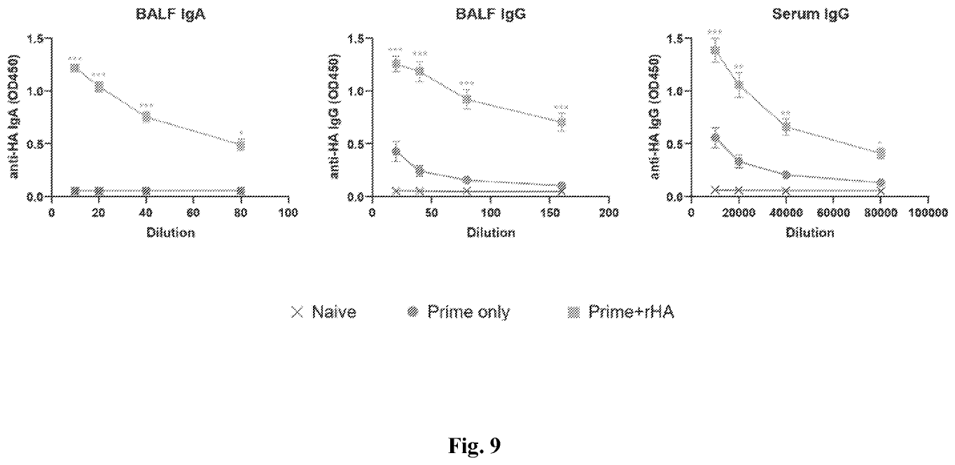

Referring to FIG. 9, the non-limiting “prime and airway boost” immunization strategy induced local mucosal IgA and systemic IgG. Comparing with both naïve mice that were not immunized and mice that only received the LNP-based “priming” immunization, the mice that received both the intramuscular “prime” immunization and the intranasal recombinant HA “boost” has significantly higher concentrations of anti-HA IgA in the BALF (left panel), as well as significantly higher concentrations of anti-HA IgG in the BALF (middle panel) and the serum (right panel).

### Example 3

### “Prime and Airway Boost” Immunization Generates Strong Memory Response

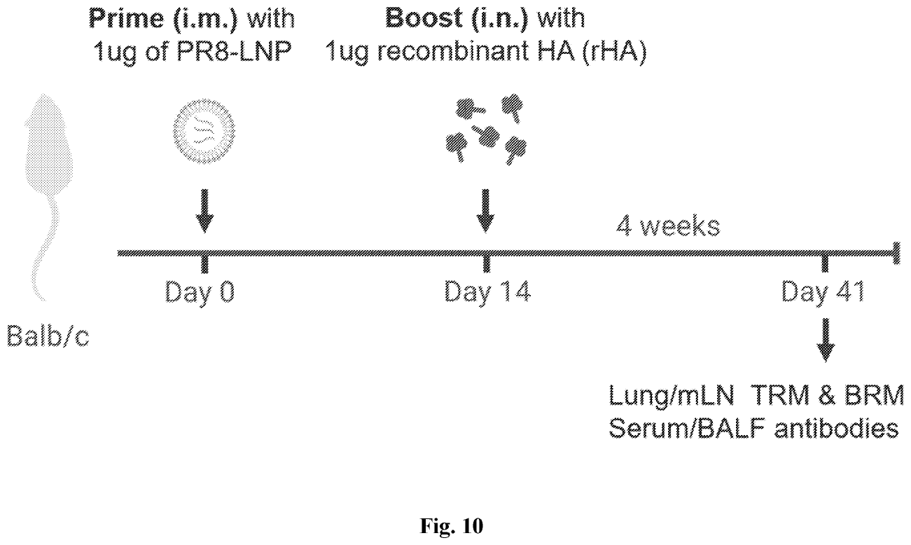

Referring to FIG. 10, at day 0, BALB/c mice were administered intramuscularly (“i.m.”) with 1 μg of a non-limiting lipid nanoparticle (LNP) based influenza vaccine, which contains mRNA encoding the hemagglutinin (HA) protein of the influenza virus stain PR8 (a strain of the H1N1 subtype) (the “prime” step). At day 14, the mice were administered intranasally (“i.n.”) with 1 μg of a recombinant HA protein of the influenza virus stain PR8 (the “boost” step). Four weeks from the boost step, resident memory T-cells (“T_{RM}”) and resident memory B-cells (“B_{RM}”) were analyzed in the samples of the lung and the mLN, and antibodies were analyzed in the samples of the serum and the BALF.

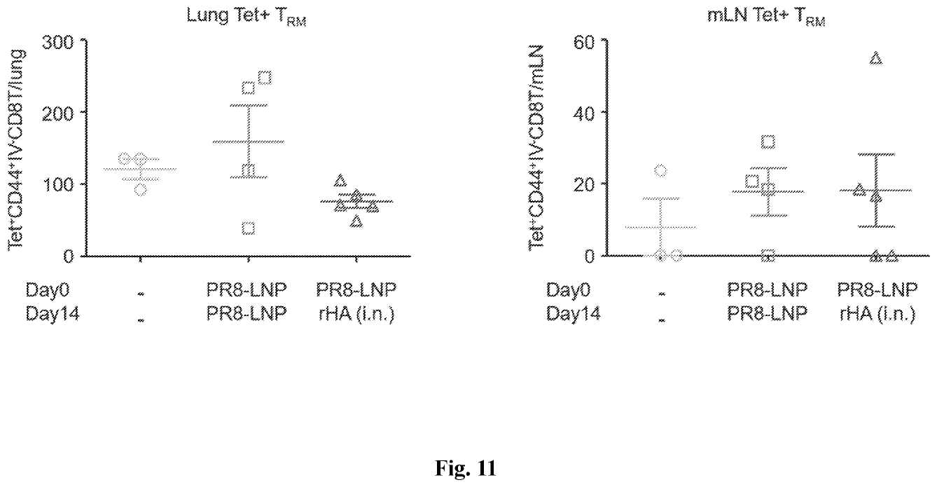

Referring to FIG. 11, the non-limiting “prime and airway boost” immunizations generated levels of antigen-specific CD8⁺ resident memory T cells at 4 weeks comparable to those generated using the PR8 HA mRNA LNP vaccine as both the “prime” and “boost.” Left panel: counts of tissue resident memory CD8 T cells in the lungs of naïve mice (left), mice primed with the LNP based influenza vaccine and boosted with the same LNP based vaccine (middle), and mice primed with the LNP based influenza vaccine and boosted with the intranasal administered recombinant HA protein (right). Right panel: counts of tissue resident memory CD8 T cells in the mLN of naïve mice (left), mice primed with the LNP based influenza vaccine and boosted with the same LNP based vaccine (middle), and mice primed with the LNP based influenza vaccine and boosted with the intranasal administered recombinant HA protein (right).

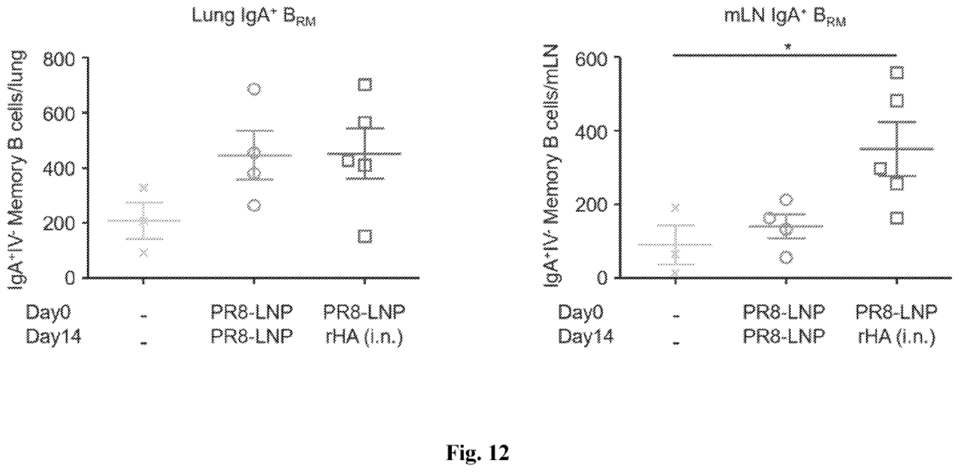

Referring to FIG. 12, 4 weeks after the non-limiting “prime and airway boost” immunizations the levels of IgA-producing tissue resident memory B cells are comparable to or higher than those generated using the PR8 HA mRNA LNP vaccine as both the “prime” and “boost.” Left panel: counts of tissue resident memory B cells that express anti-HA IgA in the lungs of naïve mice (left), mice primed with the LNP based influenza vaccine and boosted with the same LNP based vaccine (middle), and mice primed with the LNP based influenza vaccine and boosted with the intranasal administered recombinant HA protein. Right panel: counts of tissue resident memory B cells that express IgA in the mediastinal lymph nodes (mLN) of naïve mice (left), mice primed with the LNP based influenza vaccine and boosted with the same LNP based vaccine (middle), and mice primed with the LNP based influenza vaccine and boosted with the intranasal administered recombinant HA protein.

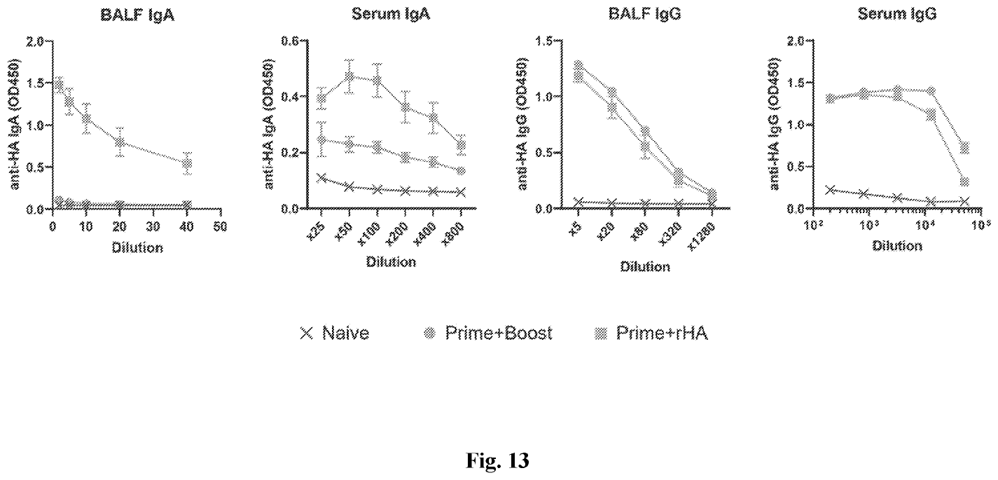

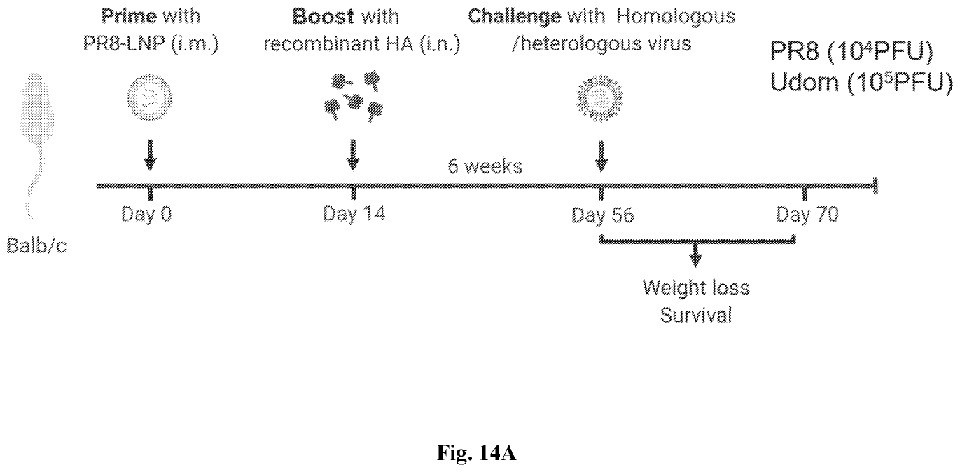

Referring to FIG. 13, the non-limiting “prime and airway boost” immunizations induced robust local and systemic antibody responses. Specifically, in comparison with naïve mice that did not receive immunization (“Naive”) or mice that received the LNP based influenza vaccine twice (“Prime+Boost”), mice that received the non-limiting “prime and airway boost” immunization illustrated in FIG. 10 (“Prime+rHA”) have significantly higher counts of anti-HA IgA in both BALF (left most panel) and serum (mid-left panel). In comparison with mice that received the LNP based influenza vaccine twice (“Prime+Boost”), mice that received the non-limiting “prime and airway boost” immunization (“Prime+rHA”) have comparable levels of anti-HA IgG in the BALF (mid right panel) and the serum (rightmost panel). It is worth noting that influenza-specific IgA has been shown to be more effective in preventing infections compared with influenza-specific IgG, and elevated IgA serum levels have been correlated with influenza vaccine efficacy (Liew et al., Eur. J. Immunol. 14, 350-356 (1984), Asahi-Ozaki et al., J. Med. Virol. 74, 328-335 (2004), and Ainai et al, Hum. Vaccin. Immunother, 9, 1962-1970 (2013))

### Example 4

### “Prime and Airway Boost” Immunization Strategy Confers Protection to Heterosubtypic and Heterologous Virus Infection

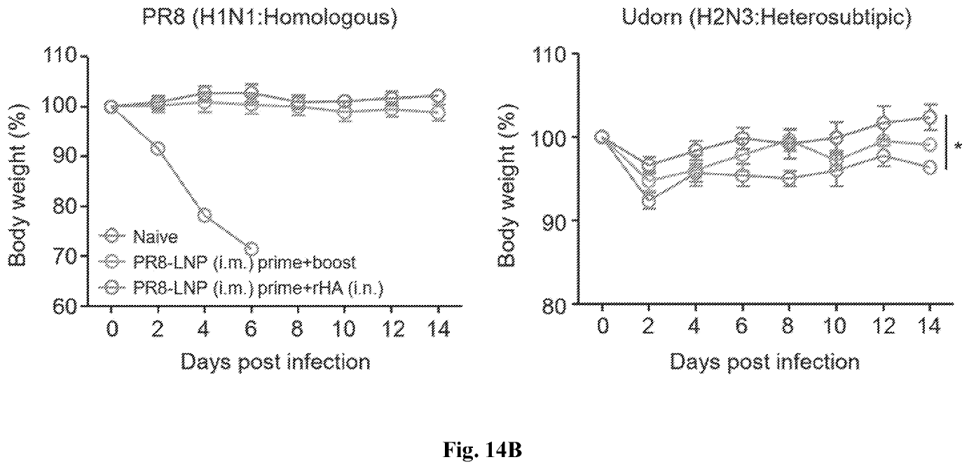

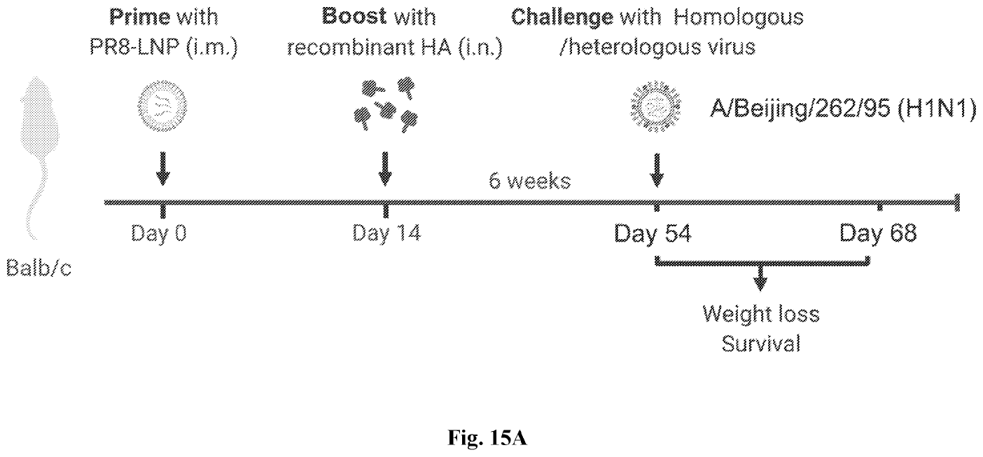

Referring to FIGS. 14A-14B, a non-limiting “prime and airway boost” immunization strategy using intranasally administered recombinant HA protein as the booster conferred better protection against heterologous viral infection than similar immunization strategies using intramuscularly an administered booster.

Referring to FIG. 14A, according to this non-limiting “prime and airway boost” strategy, BALB/c mice were administered intramuscularly with a non-limiting lipid nanoparticle (LNP) based influenza vaccine, which contains mRNA encoding the hemagglutinin (HA) protein of the influenza virus stain PR8 (a strain of the H1N1 subtype) (the “prime” step). At day 14, the animals were administered intranasally (“i.n.”) with of a recombinant HA protein of the influenza virus stain PR8 (the “boost” step). At day 56, the mice were challenged with the homologous PR8 virus (10⁴ PFU). The weight loss and survival of the mice were tracked for the next two weeks.

Referring to FIG. 14B, the non-limiting intranasal HA protein booster conferred comparable levels of protection against homologous viral infection-caused weight loss to the LNP based boost, and both of which are significantly better than non-immunization (left panel).

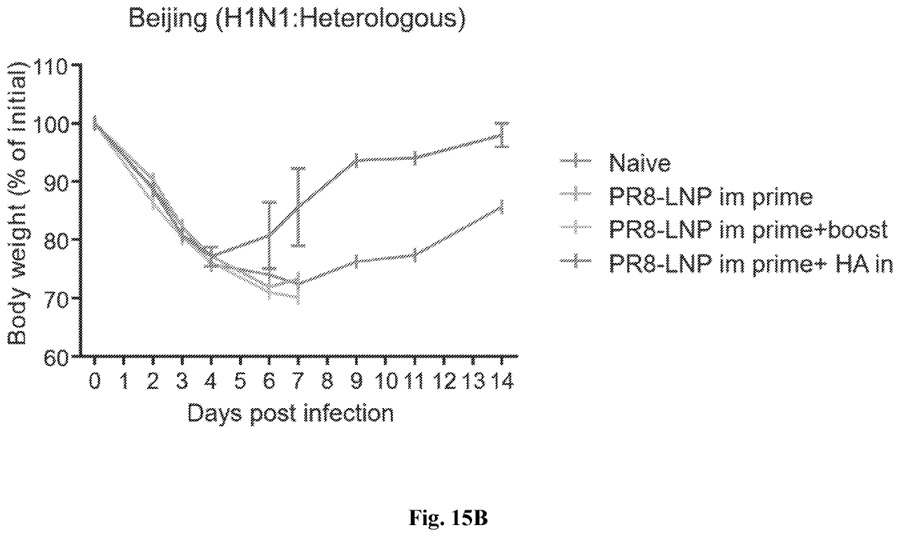

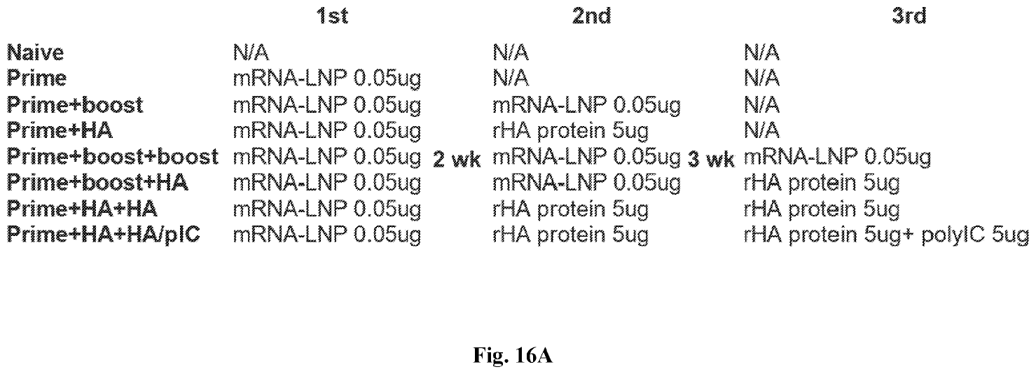

Referring FIGS. 15A-15B, the “prime and airway boost” immunization strategies using intranasally administered recombinant HA protein as the booster herein conferred better protection against heterologous viral infection than similar immunization strategies using intramuscularly administered booster.

Referring to FIG. 15A, BALB/c mice were administered intramuscularly with a non-limiting lipid nanoparticle (LNP) based influenza vaccine, which contains mRNA encoding the hemagglutinin (HA) protein of the influenza virus stain PR8 (a strain of the H1N1 subtype) (the “prime” step). At day 14, the animals were administered intranasally (“i.n.”) with of a recombinant HA protein of the influenza virus stain PR8f (the “airway boost” step). At day 54, the mice were challenged with either the homologous PR8 virus or with the heterologous influenza virial strain A/Beijing/262/95, which is of H1N1 subtype. The weight loss and survival of the mice were tracked for the next two weeks.

Referring to FIG. 15B, the non-limiting intranasal HA protein booster conferred substantially better levels of protection against the heterologous viral infection-caused weight loss than the LNP based booster, “prime” only, and non-immunization.

### Example 5

### Combining “Prime and Airway Boost” Immunization Strategy With Conventional Parenteral Vaccination Resulted in Dramatically Increased Protective Efficacies

In some embodiments, the present study discovered that combining the novel “prime and airway boost” immunization strategy with the conventional parenteral immunization strategy was able to achieve excellent protective efficacies against viral infection, which was significantly higher than protective efficacies achieved by either the “prime and airway boost” immunization strategy alone or the conventional parenteral immunization and boost strategy alone.

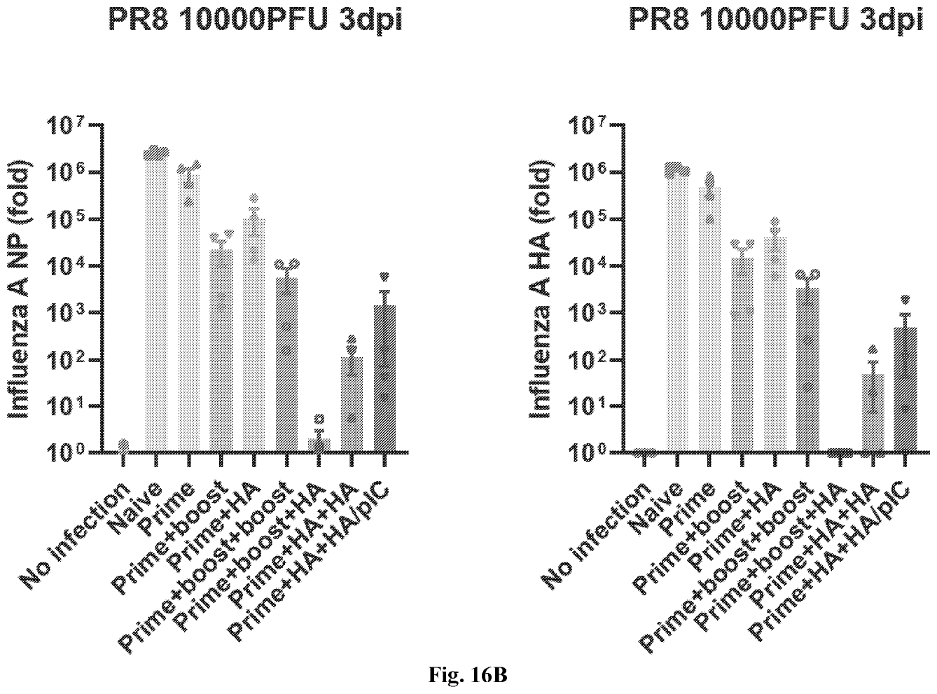

Referring to FIG. 16A, Balb/c mice were immunized with intramuscular injection of influenza mRNA lipid nanoparticle composition (“mRNA-LNP”) vaccine and/or intranasal administration of recombinant HA (“rHA”) according to various immunization strategy.

Referring to FIG. 16B, comparing with mice that were intramuscularly “primed” with mRNA-LNP and twice intramuscularly “boosted” with mRNA-LNP (“Prime+boost+boost”), mice that were intramuscularly “primed” with mRNA-LNP and twice “airway boosted” with recombinant HA protein (“Prime+HA+HA” and “Prime+HA+HA/pIC”) were better at controlling the viral load in response to PR8 influenza viral challenges.

Notably, mice that were intramuscularly “primed” with mRNA-LNP, intramuscularly “boosted” with mRNA-LNP, and then “airway boosted” with recombinant HA protein (“Prime+boost+HA”) displayed dramatically increased immunity against PR8 influenza viral challenge, to the extent that only extremely low levels of viral RNAs (nucleoprotein and HA RNA) could be detected post-infection (FIG. 16B).

### ENUMERATED EMBODIMENTS

Embodiment 1: A method of immunizing a subject against an influenza viral infection, the method comprising: parenterally administering to the subject a first composition comprising a first compound in an amount sufficient to elicit systemic T and/or B cell response(s) against a first influenza virus in the subject; and administering to the subject a second composition comprising a viral protein of a second influenza virus through an administration route comprising intranasal, inhalational, intratracheal, intrapulmonary, and intrabronchial, in an amount sufficient to result in establishment of tissue-resident T and/or B cell(s), mucosal IgA, and/or systemic IgG specific against the influenza viral infection in the subject.

Embodiment 2: A method of treating, ameliorating, and/or preventing an influenza viral infection in a subject, the method comprising: parenterally administering to the subject a first composition comprising a first compound in an amount sufficient to elicit systemic T and/or B cell response(s) against the influenza virus in the subject; and administering to the subject a second composition comprising a viral protein of a second influenza virus through an administration route comprising intranasal, inhalational, intratracheal, intrapulmonary, and intrabronchial, in an amount sufficient to result in establishment of a tissue-resident T and/or B cell(s) or mucosal IgA specific against the influenza viral infection in the subject.

Embodiment 3: The method of any one of Embodiments 1-2, further comprises parentally administering to the subject a third composition for boosting the immune response elicited by the first composition or the immune response elicited by both the first composition and the second composition.

Embodiment 4: The method of Embodiment 3, wherein the third composition is administered between the administrations of the first composition and the second composition, or is administered after the second composition in the subject.

Embodiment 5: The method of any one of Embodiments 3-4, wherein the third composition is the same as the first composition.

Embodiment 6: The method of any one of Embodiments 1-5, wherein the first influenza virus or the second influenza virus is independently an influenza A virus, an influenza B virus, an influenza C virus, or combinations thereof.

Embodiment 7: The method of any one of Embodiments 1-6, wherein a hemagglutinin protein of the first influenza virus or the second influenza virus is independently an H1 subtype, an H2 subtype, an H3 subtype, an H4 subtype, an H5 subtype, an H6 subtype, an H7 subtype, an H8 subtype, an H9 subtype, an H10 subtype, an H11 subtype, an H12 subtype, an H13 subtype, an H14 subtype, an H15 subtype, an H16 subtype, an H17 subtype, an H18 subtype, or combinations thereof.

Embodiment 8: The method of any one of Embodiments 1-7, wherein a neuraminidase protein of the first influenza virus or the second influenza virus is independently an N1 subtype, an N2 subtype, an N3 subtype, an N4 subtype, an N5 subtype, an N6 subtype, an N7 subtype, an N8 subtype, an N8 subtype, an N9 subtype, an N10 subtype, an N11 subtype, or combinations thereof.

Embodiment 9: The method of any one of Embodiments 1-8, wherein the first composition and/or the third composition comprises a vaccine against the first influenza virus.

Embodiment 10: The method Embodiment 9, wherein the vaccine against the first influenza virus comprises at least one selected from the group consisting of a mRNA-lipid nanoparticle (LNP)-based vaccine against the influenza viral infection, a viral vector vaccine against the influenza viral infection, an inactivated virus vaccine against the influenza viral infection, a viral subunit/peptide vaccine against the influenza viral infection, a virus like particle (VLP)-based vaccine against the influenza viral infection, and a DNA vaccine against the influenza viral infection.

Embodiment 11: The method of any one of Embodiments 1-10, wherein the second composition comprises a surface protein of the second influenza virus.

Embodiment 12: The method of any one of Embodiments 1-11. wherein the second composition comprises a hemagglutinin protein of the second influenza virus, a neuraminidase protein of the second influenza virus, or combinations thereof.

Embodiment 13: The method of any one of Embodiments 1-12, wherein the first composition elicits systemic T and/or B cell response(s) against a protein in the first influenza virus the same as or orthologous to the viral protein comprised in the second composition in the subject.

Embodiment 14: The method of any one of Embodiments 1-13, wherein the viral protein of the second influenza virus in the second composition is a recombinant protein, a purified protein, or both.

Embodiment 15: The method of any one of Embodiments 1-14, wherein the first influenza virus and the second influenza virus are the same.

Embodiment 16: The method of any one of Embodiments 1-14, wherein the first influenza virus and the second influenza virus are different.

Embodiment 17: The method of Embodiment 16, wherein the first influenza virus and the second influenza viruses are of the same subtype but different strains.

Embodiment 18: The method of Embodiment 16, wherein the first influenza virus and the second influenza viruses are of different subtypes.

Embodiment 19: The method of any one of Embodiments 1-18, wherein the second composition does not comprise an adjuvant.

Embodiment 20: The method of any of Embodiments 1-19, wherein the second composition comprises a pharmaceutically acceptable carrier suitable for intranasal, inhalational, intratracheal, intrapulmonary, and/or intrabronchial administration.

Embodiment 21: The method of any one of Embodiments 1-20, wherein the administration of the first composition, the administration of the second composition, and/or the administration of the third composition are separated by about 3 days to about 60 days from each other.

Embodiment 22: The method of any one of Embodiments 1-21, wherein the method results in expansion of antigen-specific, tissue-resident memory T and/or B cell response(s) against the virus in the lung, the airway or the mediastinal lymph nodes (mLNs) of the subject.

Embodiment 23: The method of any one of Embodiments 1-22, wherein the subject is a mammal, optionally a human.

Embodiment 24: A kit for immunizing a subject against an influenza viral infection, the kit comprising: a first composition for administering to the subject parenterally, the first composition comprising: a first compound for eliciting systemic T and/or B cell response(s) against a first influenza virus in the subject; and a second composition for administering to the subject through an administration route comprising intranasal, inhalational, intratracheal, intrapulmonary, and intrabronchial, the second composition comprising: a viral protein of a second influenza virus for establishment of tissue-resident T and/or B cell(s), mucosal IgA, and/or systemic IgG specific against the influenza viral infection in the subject.

Embodiment 25: A kit for treating, ameliorating, and/or preventing infection by an influenza virus in a subject, the kit comprising: a first composition for administering to the subject parenterally, the first composition comprising: a first compound for eliciting systemic T and/or B cell response(s) against a first influenza virus in the subject; and a second composition for administering to the subject through an administration route comprising intranasal, inhalational, intratracheal, intrapulmonary, and intrabronchial, the second composition comprising: a viral protein of a second influenza virus for establishment of tissue-resident T and/or B cell(s), mucosal IgA, and/or systemic IgG specific against the influenza viral infection in the subject.

Embodiment 26: The kit of any one of Embodiments 24-25, further comprising a third composition for administering to the subject parenterally, the third composition comprising a compound for boosting the immune response elicited by the first composition or the immune response elicited by both the first composition and the second composition in the subject.

Embodiment 27: The kit of Embodiment 26, wherein the third composition is for being administered between the administrations of the first composition and the second composition, or is for being administered after the second composition.

Embodiment 28: The kit of any one of Embodiments 26-27, wherein the third composition is the same as the first composition.

Embodiment 29: The kit of any one of Embodiments 24-28, wherein the first influenza virus or the second influenza virus is independently an influenza A virus, an influenza B virus, an influenza C virus, or combinations thereof.

Embodiment 30: The kit of any one of Embodiments 24-29, wherein a hemagglutinin protein of the first influenza virus or the second influenza virus is independently an H1 subtype, an H2 subtype, an H3 subtype, an H4 subtype, an H5 subtype, an H6 subtype, an H7 subtype, an H8 subtype, an H9 subtype, an H10 subtype, an H11 subtype, an H12 subtype, an H13 subtype, an H14 subtype, an H15 subtype, an H16 subtype, an H17 subtype, an H18 subtype, or combinations thereof.

Embodiment 31: The kit of any one of Embodiments 24-30, wherein a neuraminidase protein of the first influenza virus or the second influenza virus is independently an N1 subtype, an N2 subtype, an N3 subtype, an N4 subtype, an N5 subtype, an N6 subtype, an N7 subtype, an N8 subtype, an N8 subtype, an N9 subtype, an N10 subtype, an N11 subtype, or combinations thereof.

Embodiment 32: The kit of any one of Embodiments 24-31, wherein the first composition and/or the third composition comprises a vaccine against the first influenza virus.

Embodiment 33: The kit of Embodiment 32, wherein the vaccine against the first influenza virus comprises at least one selected from the group consisting of a mRNA-lipid nanoparticle (LNP)-based vaccine against the influenza viral infection, a viral vector vaccine against the influenza viral infection, an inactivated virus vaccine against the influenza viral infection, a viral subunit/peptide vaccine against the influenza viral infection, a virus like particle (VLP)-based vaccine against the influenza viral infection, and a DNA vaccine against the influenza viral infection.

Embodiment 34: The kit of any one of Embodiments 24-33, wherein the second composition comprises a surface protein of the second influenza virus.

Embodiment 35: The kit of any one of Embodiments 24-34, wherein the second composition comprises a hemagglutinin protein of the second influenza virus, a neuraminidase protein of the second influenza virus, or combinations thereof.

Embodiment 36: The kit of any one of Embodiments 24-35, wherein the first composition elicits systemic T and/or B cell response(s) against a protein in the first influenza virus the same as or orthologous to the viral protein comprised in the second composition in the subject.

Embodiment 37: The kit of any one of Embodiments 24-36, wherein the viral protein of the second influenza virus in the second composition is a recombinant protein, a purified protein, or both.

Embodiment 38: The kit of any one of Embodiments 24-37, wherein the first influenza virus and the second influenza virus are the same.

Embodiment 39: The kit of any one of Embodiments 24-37, wherein the first influenza virus and the second influenza virus are different.

Embodiment 40: The kit of Embodiment 39, wherein the first influenza virus and the second influenza viruses are of the same subtype but different strains.

Embodiment 41: The kit of Embodiment 39, wherein the first influenza virus and the second influenza viruses are of different subtypes.

Embodiment 42: The kit of any one of Embodiments 24-41, wherein the second composition does not comprise an adjuvant.

Embodiment 43: The kit of any of Embodiments 24-42, wherein the second composition comprises a pharmaceutically acceptable carrier suitable for intranasal, inhalational, intratracheal, intrapulmonary, and/or intrabronchial administration.

Embodiment 44: The kit of any one of Embodiments 24-43, wherein the administration of the first composition and the second composition results in expansion of antigen-specific, tissue-resident memory T and/or B cell response(s) against the virus in the lung, the airway or the mediastinal lymph nodes (mLNs) of the subject.

Embodiment 45: The kit of any one of Embodiments 24-44, wherein the subject is a mammal, optionally a human.

Embodiment 46: A method of boosting an existing immunity against an influenza viral infection in a subject, wherein the existing immunity is acquired via a parenteral administration to the subject a first composition comprising a first compound in an amount sufficient to elicit systemic T and/or B cell response(s) against a first influenza virus in the subject, the method comprising: administering to the subject a second composition comprising a viral protein of an influenza virus through an administration route comprising intranasal, inhalational, intratracheal, intrapulmonary, and intrabronchial, in an amount sufficient to result in establishment of tissue-resident T and/or B cell(s), mucosal IgA, and/or systemic IgG specific against the influenza viral infection in the subject.

Embodiment 47: The method of Embodiment 46, further comprises parentally administering to the subject a third composition for boosting the immune response elicited by the first composition or the immune response elicited by both the first composition and the second composition.

Embodiment 48: The method of Embodiment 47, wherein the third composition is administered before or after the second composition.

Embodiment 49: The method of any one of Embodiments 47-48, wherein the third composition is the same as the first composition.

Embodiment 50: The method of Embodiment 49, wherein the first influenza virus or the second influenza virus is independently an influenza A virus, an influenza B virus, an influenza C virus, or combinations thereof.

Embodiment 51: The method of any one of Embodiments 46-50, wherein a hemagglutinin protein of the first influenza virus or the second influenza virus is independently an H1subtype, an H2 subtype, an H3 subtype, an H4 subtype, an H5 subtype, an H6 subtype, an H7 subtype, an H8 subtype, an H9 subtype, an H10 subtype, an H11 subtype, an H12 subtype, an H13 subtype, an H14 subtype, an H15 subtype, an H16 subtype, an H17 subtype, an H18 subtype, or combinations thereof.

Embodiment 52: The method of any one of Embodiments 46-51, wherein a neuraminidase protein of the first influenza virus or the second influenza virus is independently an N1 subtype, an N2 subtype, an N3 subtype, an N4 subtype, an N5 subtype, an N6 subtype, an N7 subtype, an N8 subtype, an N8 subtype, an N9 subtype, an N10 subtype, an N11 subtype, or combinations thereof.

Embodiment 53: The method of any one of Embodiments 46-52, wherein the first composition comprises a vaccine against the first influenza virus.

Embodiment 54: The method Embodiment 53, wherein the vaccine against the first influenza virus comprises at least one selected from the group consisting of a mRNA-lipid nanoparticle (LNP)-based vaccine against the influenza viral infection, a viral vector vaccine against the influenza viral infection, an inactivated virus vaccine against the influenza viral infection, a viral subunit/peptide vaccine against the influenza viral infection, a virus like particle (VLP)-based vaccine against the influenza viral infection, and a DNA vaccine against the influenza viral infection.

Embodiment 55: The method of any one of Embodiments 46-54, wherein the second composition comprises a surface protein of the second influenza virus.

Embodiment 56: The method of any one of Embodiments 46-55, wherein the second composition comprises a hemagglutinin protein of the second influenza virus, a neuraminidase protein of the second influenza virus, or combinations thereof.

Embodiment 57: The method of any one of Embodiments 46-56, wherein the first composition elicits systemic T and/or B cell response(s) against a protein in the first influenza virus the same as or orthologous to the viral protein comprised in the second composition in the subject.

Embodiment 58: The method of any one of Embodiments 46-57, wherein the viral protein of the second influenza virus in the second composition is a recombinant protein, a purified protein, or both.

Embodiment 59: The method of any one of Embodiment 46-58, wherein the first influenza virus and the second influenza virus are the same.

Embodiment 60: The method of any one of Embodiment 46-58, wherein the first influenza virus and the second influenza virus are different.

Embodiment 61: The method of Embodiment 60, wherein the first influenza virus and the second influenza viruses are of the same subtype but different strains.

Embodiment 62: The method of Embodiment 60, wherein the first influenza virus and the second influenza viruses are of different subtypes.

Embodiment 63: The method of any one of Embodiments 46-62, wherein the second composition does not comprise an adjuvant.

Embodiment 64: The method of any of Embodiments 46-63, wherein the second composition comprises a pharmaceutically acceptable carrier suitable for intranasal, inhalational, intratracheal, intrapulmonary, and/or intrabronchial administration.

Embodiment 65: The method of any one of Embodiments 46-64, wherein the administration of the first composition, the second composition and/or the third composition are separated from each other by about 3 days to about 60 days.

Embodiment 66: The method of any one of Embodiments 46-65, wherein the method results in expansion of antigen-specific, tissue-resident memory T and/or B cell response(s) against the virus in the lung, the airway or the mediastinal lymph nodes (mLNs) of the subject.

Embodiment 67: The method of any one of Embodiments 46-66, wherein the subject is a mammal, such as a human.

Embodiment 68: A composition for boosting an existing immunity against an influenza viral infection in a subject, the composition comprising: a viral protein of an influenza virus; and a pharmaceutically acceptable carrier suitable for intranasal, inhalational, intratracheal, intrapulmonary, and/or intrabronchial administration of the protein of the influenza virus.

Embodiment 69: The composition of Embodiment 68, wherein the influenza virus is an influenza A virus, an influenza B virus, an influenza C virus, or combinations thereof.

Embodiment 70: The composition of any one of Embodiments 67-69, wherein a hemagglutinin protein of the influenza virus is an H1 subtype, an H2 subtype, an H3 subtype, an H4 subtype, an H5 subtype, an H6 subtype, an H7 subtype, an H8 subtype, an H9 subtype, an H10 subtype, an H11 subtype, an H12 subtype, an H13 subtype, an H14 subtype, an H15 subtype, an H16 subtype, an H17 subtype, an H18 subtype, or combinations thereof.

Embodiment 71: The composition of any one of Embodiments 67-70, wherein a neuraminidase protein of the influenza virus is an N1 subtype, an N2 subtype, an N3 subtype, an N4 subtype, an N5 subtype, an N6 subtype, an N7 subtype, an N8 subtype, an N8 subtype, an N9 subtype, an N10 subtype, an N11 subtype, or combinations thereof.

Embodiment 72: The composition of any one of Embodiments 67-71, wherein the viral protein is a surface protein of the influenza virus.

Embodiment 73: The composition of any one of Embodiments 67-72, wherein the viral protein is a hemagglutinin protein of the influenza virus, a neuraminidase protein of the influenza virus, or combinations thereof.

Embodiment 74: The composition of any one of Embodiments 67-73, wherein the viral protein is a recombinant protein, a purified protein, or both.

Embodiment 75: The composition of any one of Embodiments 67-74, wherein the composition does not comprise an adjuvant.

Embodiment 76: The composition of any one of Embodiments 67-75, further comprising a pharmaceutically acceptable carrier suitable for intranasal, inhalational, intratracheal, intrapulmonary, and/or intrabronchial administration.

Embodiment 77: The composition of any one of Embodiments 67-76, wherein the existing immunity against the influenza viral infection in the subject was acquired response to a parenterally administered influenza vaccination against influenza viral infection.

Embodiment 78: The composition of any one of Embodiment 67-77, wherein the subject is a mammal, optionally a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A method of immunizing a subject against a first influenza viral infection, or treating, ameliorating, or preventing a first influenza viral infection in a subject,
the method comprising:
parenterally administering to the subject a first composition comprising a first compound in an amount sufficient to elicit systemic T and/or B cell response(s) against the first influenza virus in the subject; and
administering to the subject a second composition comprising a viral protein of a second influenza virus through an administration route comprising intranasal, inhalational, intratracheal, intrapulmonary, and intrabronchial, in an amount sufficient to result in establishment of tissue-resident T cell(s), tissue-resident B cell(s), mucosal IgA, systemic IgG specific against the influenza viral infection in the subject.

2. (canceled)

3. The method of claim 1, which further comprises parentally administering to the subject a third composition for boosting the immune response elicited by the first composition or the immune response elicited by both the first composition and the second composition.

4. The method of claim 3, wherein at least one of the following applies:
the third composition is administered between the administrations of the first composition and the second composition, or is administered after the second composition in the subject;
the third composition is the same as the first composition;
the third composition comprises a vaccine against the first influenza virus;
the administration of the first composition, the administration of the second composition, or the administration of the third composition are separated by about 3 days to about 60 days from each other.

5. (canceled)

6. The method of claim 1, wherein at least one of the following applies:
the first influenza virus or the second influenza virus is independently an influenza A virus, an influenza B virus, an influenza C virus, or combinations thereof;
a hemagglutinin protein of the first influenza virus or the second influenza virus is independently an H1 subtype, an H2 subtype, an H3 subtype, an H4 subtype, an H5 subtype, an H6 subtype, an H7 subtype, an H8 subtype, an H9 subtype, an H10 subtype, an H11 subtype, an H12 subtype, an H13 subtype, an H14 subtype, an H15 subtype, an H16 subtype, an H17 subtype, an H18 subtype, or combinations thereof;
a neuraminidase protein of the first influenza virus or the second influenza virus is independently an N1 subtype, an N2 subtype, an N3 subtype, an N4 subtype, an N5 subtype, an N6 subtype, an N7 subtype, an N8 subtype, an N8 subtype, an N9 subtype, an N10 subtype, an N11 subtype, or combinations thereof.

7-8. (canceled)

9. The method of claim 1, wherein the first composition comprises a vaccine against the first influenza virus,
optionally wherein the vaccine against the first influenza virus comprises at least one selected from the group consisting of a mRNA-lipid nanoparticle (LNP)-based vaccine against the influenza viral infection, a viral vector vaccine against the influenza viral infection, an inactivated virus vaccine against the influenza viral infection, a viral subunit/peptide vaccine against the influenza viral infection, a virus like particle (VLP)-based vaccine against the influenza viral infection, and a DNA vaccine against the influenza viral infection.

10. (canceled)

11. The method of claim 1, wherein the second composition comprises a surface protein of the second influenza virus;
optionally the second composition comprises a hemagglutinin protein of the second influenza virus, a neuraminidase protein of the second influenza virus, or combinations thereof.

12. (canceled)

13. The method of claim 1, wherein the first composition elicits systemic T or B cell response(s) against a protein in the first influenza virus the same as or orthologous to the viral protein comprised in the second composition in the subject.

14. The method of claim 1, wherein at least one of the following applies:
the viral protein of the second influenza virus in the second composition is a recombinant protein, a purified protein, or both;
the first influenza virus and the second influenza virus are the same;
the first influenza virus and the second influenza virus are different;
the first influenza virus and the second influenza viruses are of the same subtype but different strains;
the first influenza virus and the second influenza viruses are of different subtypes;
the second composition does not comprise an adjuvant;
the second composition comprises a pharmaceutically acceptable carrier suitable for intranasal, inhalational, intratracheal, intrapulmonary, and/or intrabronchial administration;
the administration of the first composition and the administration of the second composition are separated by about 3 days to about 60 days from each other;
the subject is a mammal, optionally a human.

15-20. (canceled)

21. The method of claim 3, wherein the administration of the first composition; or the administration of the second composition, are separated by about 3 days to about 60 days from each other.

22. The method of claim 1, wherein the method results in expansion of antigen-specific, tissue-resident memory T or B cell response(s) against the virus in the lung, the airway or the mediastinal lymph nodes (mLNs) of the subject.

23. (canceled)

24. A kit for immunizing a subject against an a first influenza viral infection, or for treating, ameliorating, or preventing infection by a first influenza virus in a subject, the kit comprising:
a first composition for administering to the subject parenterally, the first composition comprising: a first compound for eliciting systemic T or B cell response(s) against a first influenza virus in the subject; and
a second composition for administering to the subject through an administration route comprising intranasal, inhalational, intratracheal, intrapulmonary, and intrabronchial, the second composition comprising: a viral protein of a second influenza virus for establishment of tissue-resident T cell(s), tissue-resident B cell(s), mucosal IgA, or systemic IgG specific against the influenza viral infection in the subject.

25. (canceled)

26. The kit of claim 24, further comprising a third composition for administering to the subject parenterally, the third composition comprising a compound for boosting the immune response elicited by the first composition or the immune response elicited by both the first composition and the second composition in the subject.

27. The kit of claim 26, wherein at least one of the following applies:
the third composition is for being administered between the administrations of the first composition and the second composition, or is for being administered after the second composition;
the third composition comprises a vaccine against the first influenza virus;
the third composition is the same as the first composition.

28. (canceled)

29. The kit of claim 24, wherein at least one of the following applies:
the first influenza virus or the second influenza virus is independently an influenza A virus, an influenza B virus, an influenza C virus, or combinations thereof;
a hemagglutinin protein of the first influenza virus or the second influenza virus is independently an H1 subtype, an H2 subtype, an H3 subtype, an H4 subtype, an H5 subtype, an H6 subtype, an H7 subtype, an H8 subtype, an H9 subtype, an H10 subtype, an H11 subtype, an H12 subtype, an H13 subtype, an H14 subtype, an H15 subtype, an H16 subtype, an H17 subtype, an H18 subtype, or combinations thereof;
a neuraminidase protein of the first influenza virus or the second influenza virus is independently an N1 subtype, an N2 subtype, an N3 subtype, an N4 subtype, an N5 subtype, an N6 subtype, an N7 subtype, an N8 subtype, an N8 subtype, an N9 subtype, an N10 subtype, an N11 subtype, or combinations thereof.

30-31. (canceled)

32. The kit of claim 24, wherein the first composition comprises a vaccine against the first influenza virus;
optionally wherein the vaccine against the first influenza virus comprises at least one selected from the group consisting of a mRNA-lipid nanoparticle (LNP)-based vaccine against the influenza viral infection, a viral vector vaccine against the influenza viral infection, an inactivated virus vaccine against the influenza viral infection, a viral subunit/peptide vaccine against the influenza viral infection, a virus like particle (VLP)-based vaccine against the influenza viral infection, and a DNA vaccine against the influenza viral infection.

33. (canceled)

34. The kit of claim 24, wherein at least one of the following applies:
the second composition comprises a surface protein of the second influenza virus;
the second composition comprises a hemagglutinin protein of the second influenza virus, a neuraminidase protein of the second influenza virus, or combinations thereof;
the first composition elicits systemic T or B cell response(s) against a protein in the first influenza virus the same as or orthologous to the viral protein comprised in the second composition in the subject;
the viral protein of the second influenza virus in the second composition is a recombinant protein, a purified protein, or both;
the first influenza virus and the second influenza virus are the same;
the first influenza virus and the second influenza virus are different;
the first influenza virus and the second influenza viruses are of the same subtype but different strains;
the first influenza virus and the second influenza viruses are of different subtypes;
the second composition does not comprise an adjuvant;
the second composition comprises a pharmaceutically acceptable carrier suitable for intranasal, inhalational, intratracheal, intrapulmonary, and/or intrabronchial administration;
the administration of the first composition and the second composition results in expansion of antigen-specific, tissue-resident memory T or B cell response(s) against the virus in the lung, the airway or the mediastinal lymph nodes (mLNs) of the subject;
the subject is a mammal, optionally a human.

35-45. (canceled)

46. A method of boosting an existing immunity against an influenza viral infection in a subject, wherein the existing immunity is acquired via a parenteral administration to the subject a first composition comprising a first compound in an amount sufficient to elicit systemic T or B cell response(s) against a first influenza virus in the subject, the method comprising:
administering to the subject a second composition comprising a viral protein of an influenza virus through an administration route comprising intranasal, inhalational, intratracheal, intrapulmonary, and intrabronchial, in an amount sufficient to result in establishment of tissue-resident T cell(s), tissue-resident B cell(s), mucosal IgA, or systemic IgG specific against the influenza viral infection in the subject.

47. The method of claim 46, which further comprises parentally administering to the subject a third composition for boosting the immune response elicited by the first composition or the immune response elicited by both the first composition and the second composition.

48. The method of claim 47, wherein at least one of the following applies:
the third composition is administered before or after the second composition;
the administration of the first composition, the second composition, or the third composition are separated from each other by about 3 days to about 60 days;
the third composition is the same as the first composition.

49. (canceled)

50. The method of claim 46, wherein at least one of the following applies:
the first influenza virus or the second influenza virus is independently an influenza A virus, an influenza B virus, an influenza C virus, or combinations thereof;
a hemagglutinin protein of the first influenza virus or the second influenza virus is independently an H1 subtype, an H2 subtype, an H3 subtype, an H4 subtype, an H5 subtype, an H6 subtype, an H7 subtype, an H8 subtype, an H9 subtype, an H10 subtype, an H11 subtype, an H12 subtype, an H13 subtype, an H14 subtype, an H15 subtype, an H16 subtype, an H17 subtype, an H18 subtype, or combinations thereof;
a neuraminidase protein of the first influenza virus or the second influenza virus is independently an N1 subtype, an N2 subtype, an N3 subtype, an N4 subtype, an N5 subtype, an N6 subtype, an N7 subtype, an N8 subtype, an N8 subtype, an N9 subtype, an N10 subtype, an N11 subtype, or combinations thereof.

51-52. (canceled)

53. The method of claim 46, wherein the first composition comprises a vaccine against the first influenza virus;
optionally wherein the vaccine against the first influenza virus comprises at least one selected from the group consisting of a mRNA-lipid nanoparticle (LNP)-based vaccine against the influenza viral infection, a viral vector vaccine against the influenza viral infection, an inactivated virus vaccine against the influenza viral infection, a viral subunit/peptide vaccine against the influenza viral infection, a virus like particle (VLP)-based vaccine against the influenza viral infection, and a DNA vaccine against the influenza viral infection.

54. (canceled)

55. The method of claim 46, wherein the second composition comprises a surface protein of the second influenza virus;
optionally wherein the second composition comprises a hemagglutinin protein of the second influenza virus, a neuraminidase protein of the second influenza virus, or combinations thereof.

56. (canceled)

57. The method of claim 46, wherein at least one of the following applies:
the first composition elicits systemic T or B cell response(s) against a protein in the first influenza virus the same as or orthologous to the viral protein comprised in the second composition in the subject:
the viral protein of the second influenza virus in the second composition is a recombinant protein, a purified protein, or both;
the first influenza virus and the second influenza virus are the same.
the first influenza virus and the second influenza virus are different;
the first influenza virus and the second influenza viruses are of the same subtype but different strains;
the first influenza virus and the second influenza viruses are of different subtypes;
the second composition does not comprise an adjuvant;
the second composition comprises a pharmaceutically acceptable carrier suitable for intranasal, inhalational, intratracheal, intrapulmonary, and/or intrabronchial administration;
the method results in expansion of antigen-specific, tissue-resident memory T or B cell response(s) against the virus in the lung, the airway or the mediastinal lymph nodes (mLNs) of the subject;
the subject is a mammal, such as a human.

58-64. (canceled)

65. The method of claim 46, wherein the administration of the first composition; or the second composition and/or the third composition are separated from each other by about 3 days to about 60 days.

66-67. (canceled)

68. A composition for boosting an existing immunity against an influenza viral infection in a subject, the composition comprising:
a viral protein of an influenza virus; and
a pharmaceutically acceptable carrier suitable for intranasal, inhalational, intratracheal, intrapulmonary, or intrabronchial administration of the protein of the influenza virus;
optionally wherein the existing immunity against the influenza viral infection in the subject was acquired response to a parenterally administered influenza vaccination against influenza viral infection.

69. The composition of claim 68, wherein at least one of the following applies:
the influenza virus is an influenza A virus, an influenza B virus, an influenza C virus, or combinations thereof;
a hemagglutinin protein of the influenza virus is an H1 subtype, an H2 subtype, an H3 subtype, an H4 subtype, an H5 subtype, an H6 subtype, an H7 subtype, an H8 subtype, an H9 subtype, an H10 subtype, an H11 subtype, an H12 subtype, an H13 subtype, an H14 subtype, an H15 subtype, an H16 subtype, an H17 subtype, an H18 subtype, or combinations thereof;
a neuraminidase protein of the influenza virus is an N1 subtype, an N2 subtype, an N3 subtype, an N4 subtype, an N5 subtype, an N6 subtype, an N7 subtype, an N8 subtype, an N8 subtype, an N9 subtype, an N10 subtype, an N11 subtype, or combinations thereof;
the viral protein is a surface protein of the influenza virus;
optionally the viral protein is a hemagglutinin protein of the influenza virus, a neuraminidase protein of the influenza virus, or combinations thereof;
the viral protein is a recombinant protein, a purified protein, or both;
the composition does not comprise an adjuvant;
the composition comprises a pharmaceutically acceptable carrier suitable for intranasal, inhalational, intratracheal, intrapulmonary, and/or intrabronchial administration;
the subject is a mammal, optionally a human.

70-78. (canceled)
